(19) United States
(12) Patent Application Publication
(10) Pub. No.: US 2024/0375376 A1
(43) Pub. Date: Nov. 14, 2024

(54) ABSORBENT ARTICLES WITH BONDED STRETCH LAMINATES

(71) Applicant: The Procter & Gamble Company, Cincinnati, OH (US)

(72) Inventors: Tanner Laurie WILLIAMS, Loveland, OH (US); Astrid Annette SHEEHAN, Symmes Township, OH (US)

(21) Appl. No.: 18/660,391

(22) Filed: May 10, 2024

Related U.S. Application Data
(60) Provisional application No. 63/501,476, filed on May 11, 2023.

(51) Int. Cl. B32B 7/14; B32B 5/02; B32B 25/10

(52) U.S. Cl. CPC B32B 7/14; B32B 5/022; B32B 25/10; B32B 2307/7375; B32B 2437/00

(57) ABSTRACT

A stretch laminate for an absorbent article. The stretch laminate includes a nonwoven substrate and an elastomeric film joined to the nonwoven substrate by a plurality of bonds. These bonds may be ultrasonic bonds or some combination of mechanical bonds, including ultrasonic bonds. The plurality of bonds form a bond pattern including a plurality of bond site clusters. Each of the bond site clusters includes a first bond having a longest bond dimension (D) and a second bond adjacent to the first bond. The Bond Separation Angle between the first bond and the second bond is from 0° up to and including 35° and the Bond Separation Distance between the first and second bond is less than 2.1D. Further, the bond site clusters may have a cluster dimension of less than about 5 mm and/or a cluster separation distance of at least about 2.1D

### CROSS REFERENCE TO RELATED APPLICATION

This application claims the benefit of U.S. Provisional Application No. 63/501,476, filed May 11, 2023, the entirety of which is incorporated herein by reference.

### FIELD

The present disclosure relates generally to of absorbent articles and more specifically to absorbent articles comprising mechanically bonded stretch laminates having a bond pattern including a plurality of bond site clusters.

### BACKGROUND

Manufacturers of disposable absorbent articles invest in multiple brands, tiers, and size lineups despite the added complexity. They do so to better meet the broad and ever evolving range of consumer aspirations over the lifecycle of their babies along with affordable economics. It therefore becomes important to offer product features that enable differentiation across sizes and tiers both for overt functional benefit as well as noticeable cues that signal underlying performance differences. Consumers, however, have always been known to multitask, and in modern times, they are noticed to be even less attentive and less sensitive to differences even with ordinary household products with which they interact regularly. This makes it essential for manufacturers to increase their investment in noticeability.

One of the key product features of disposable absorbent articles, such as diapers and pants, is the fundamental need for comfort and fit. The latter is judged at both initial application as well as during wear time and is normally achieved through product features that are extensible (i.e., stretch). One way in which manufacturers attempt to balance the competing interests of proper fit and variation in body type is through the use of expandable materials. One such group of materials is known as stretch laminates. As the name suggests, these materials are actually composites of individual components that are laminated or bonded together and provide varying degrees of stretch.

In recent years, commercialization of ultrasonically bonded laminates has gained favor by the absorbent article industry. These ultrasonically bonded laminates are simpler in construction and favorable to cost and complexity because they can eliminate the need for adhesives, which require relatively more hardware, frequent cleaning, and contribute to ongoing cost and product malodor. Importantly, these simpler stretch laminates are subject to the same distinctiveness requirements as any other product function or component.

The ultrasonically bonded laminates may be used as front or back cars in taped diapers or as front or back side panels in pants, for example. The front or back cars and side panels require different characteristics to fit across brands, tiers and size lineups. Further, these bonded laminates need to be distinctive to the consumer, such that functionality and/or aesthetics are communicated therethrough, without sacrificing performance. As such there is a need to improve design flexibility to support brands, tiers, and size lineups without incurring complexity and sacrificing performance.

More specifically, these bonded stretch laminate need to be comfortable and breathable. Due to the type of materials that used to form stretch laminates, breathability and comfort is important to maintain. These stretch laminate are placed adjacent to the users skin and, thus, the ability for air to circulate through the stretch laminate is necessary.

The discussion of shortcomings and needs existing in the field prior to the present disclosure is in no way an admission that such shortcomings and needs were recognized by those skilled in the art prior to the present disclosure.

### SUMMARY

In some embodiments, a stretch laminate for an absorbent article may include a nonwoven substrate and an elastomeric film joined to the nonwoven substrate by a plurality of ultrasonic bonds. The plurality of ultrasonic bonds form a bond pattern. The bond pattern includes a plurality of bond site clusters. Each of the bond site clusters include a first bond having a longest bond dimension (D) and a second bond adjacent to the first bond. A Bond Separation Distance between the first and second bond may be less than 2.1D and a Bond Separation Angle between the first bond and the second bond may be from 0° up to and including 35° from the bond longitudinal axis. The first bond has a first outermost point and the second bond has a second outermost point, such that a longitudinal cluster length measured in a direction substantially perpendicular to the stretch direction from the first outermost point to the second outermost point is less than about 5 mm. Stated another way, the cluster dimension is less than about 5 mm.

In some embodiments, an absorbent article includes a stretch laminate. The stretch laminate includes a nonwoven material and an elastomeric film joined to the nonwoven material by a plurality of ultrasonic bonds. The plurality of ultrasonic bonds form a bond pattern. The plurality of ultrasonic bonds include a plurality of individual bonds each having a longest bond dimension, D, in the range of about 0.6 mm to about 1 mm. The plurality of individual bonds include a plurality of bond site clusters. Each of the bond site clusters includes a first bond and a second bond adjacent to the first bond. The Bond Separation Distance between the first and second bond is less than 2.1D and the Bond Separation Angle between the first bond and the second bond is from 0° up to and including 35° from the bond longitudinal axis. Further, each of the plurality of bond site clusters is separated by a cluster separation distance. The cluster separation distance is at least about 2.1D.

In some embodiments, an absorbent article includes a stretch laminate. The stretch laminate includes a nonwoven material and an elastomeric film joined to the nonwoven material by a plurality of ultrasonic bonds. The plurality of ultrasonic bonds form a bond pattern. The plurality of ultrasonic bonds include a plurality of individual bonds each having a longest bond dimension, D, in the range of less than about 2.0 mm. The plurality of individual bonds include a first bond having a longest bond dimension, D, and a second bond adjacent to the first bond. A Bond Separation Distance and a Bond Separation Angle exist between the first bond and the second bond. The Bond Separation Distance between the first and second bond is less than 2.1D and the Bond Separation Angle between the first bond and the second bond is from 0° up to and including 35° from the bond longitudinal axis. Further, each of the bond site clusters are configured to form a break in the elastomeric film when the stretch laminate is stretched greater than about 15 mm in the stretch direction.

These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures.

FIG. 1 is a plan view of an example absorbent article in the form of a taped diaper, garment-facing surface facing the viewer, in a flat laid-out state.

FIG. 2 is a plan view of the example absorbent article of FIG. 1, wearer-facing surface facing the viewer, in a flat laid-out state.

FIG. 3 is a front perspective view of the absorbent article of FIGS. 1 and 2 in a fastened position.

FIG. 4 is a front perspective view of an absorbent article in the form of a pant.

FIG. 5 is a rear perspective view of the absorbent article of FIG. 4.

FIG. 6 is a plan view of the absorbent article of FIG. 4, laid flat, with a garment-facing surface facing the viewer.

FIG. 7 is a cross-sectional view of the absorbent article taken about line 7-7 of FIG. 6.

FIG. 8 is a cross-sectional view of the absorbent article taken about line 8-8 of FIG. 6.

FIG. 9 is a plan view of an example absorbent core or an absorbent article.

FIG. 10 is a cross-sectional view, taken about line 10-10, of the absorbent core of FIG. 9.

FIG. 11 is a cross-sectional view, taken about line 11-11, of the absorbent core of FIG. 9.

FIGS. 12A-F are a cross-sectional views of various stretch laminates.

FIG. 13A is a SEM photomicrograph showing a cross-sectional view of a portion of an elastomeric film that has not been pre-activated.

FIG. 13B is a magnified version of the SEM photomicrograph of FIG. 13A.

FIG. 14A is a SEM photomicrograph showing a cross-sectional view of a portion of a pre-activated elastomeric film.

FIG. 14B is a magnified version of the SEM photomicrograph of FIG. 14A.

FIG. 15 is a transmitted light photomicrograph of a top view of a portion of an elastomeric film that has not been pre-activated.

FIG. 16 is a transmitted light photomicrograph of a top view of a portion of a pre-activated elastomeric film illustrating activation stripes.

FIG. 17 is a schematic illustration of a continuous process for making a stretch laminate according to the present disclosure.

FIG. 18 is a schematic illustration of a plurality of individual ultrasonic bonds shapes.

FIG. 19 is a schematic illustration of an open cell bond pattern.

FIGS. 20 and 21 are schematic illustrations of laminates having bond patterns with different bond densities.

FIGS. 22A and 22B are plan views of an exemplary side members comprising laminates having more than one bond pattern.

FIG. 23 is a schematic illustration of a bond and the Bond Separation Angles and the Bond Separation Distance with respect to that bond.

FIG. 24 is a schematic illustration of a bond and two adjacent bonds and the position of the two adjacent bonds with respect to the bond.

FIG. 25 is a schematic illustration of a bond and two adjacent bonds and the position of the two adjacent bonds with respect to the bond.

FIGS. 26A-26C are schematic illustrations of bond patterns and the Bond Separation Angles and Bond Separation Distances for one or more identified bonds.

FIGS. 27A-27C are schematic illustrations of various bond shapes and the longest dimension for each of these bond shapes.

FIG. 28A is a schematic illustration of determining the Bond Separation Angles and Bond Separation Distance for a bond.

FIGS. 28B-28D are a schematic illustration of the Bond Separation Angles and Bond Separation Distances for various, non-circular bond shapes.

FIGS. 29A-29E illustrate various bond site clusters.

FIG. 30 is a schematic illustration of a stretch laminate being subject to a lateral pull force.

FIGS. 31A-31C is a schematic illustration of a stretch laminate with various types of frangible bond sites after being subjected to a lateral pull force.

FIGS. 32A-32F are schematic illustrations showing a variety of primary bond patterns for the nonwoven materials that may be employed according to various embodiments.

FIG. 33 is a schematic illustration of adjacent bonds and features relevant for determining the Bond Separation Distance.

FIG. 34 is a schematic illustration of adjacent bonds and the features relevant for determining the Bond Separation Angle.

FIG. 35A is a schematic illustration showing an exemplary back ear of an absorbent article, identifying features relevant for the Tensile Test.

FIG. 35B is a schematic illustration showing a side view exemplary back ear of an absorbent article, identifying features relevant for the Tensile Test.

FIG. 36A is a schematic illustration of a partial absorbent article having an ear attached thereto and the relevant cut lines for the Back Ear Hang Time Test.

FIG. 36B is a schematic illustration of a clamp attachment relevant to the Back Ear Hang Time Test.

FIG. 36C is a photograph of an exemplary back ear including ruptures relevant to the Back Ear Hang Time Test.

FIGS. 37A-37B are schematic illustrations of an exemplary side member of an absorbent article and identifying features relevant for a side member subject to the Extension Test and the Tensile Test Method.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent. “Absorbent article” refers to devices that absorb and contain liquid, and more specifically, refers to devices that are placed against or in proximity to the body of the wearer to absorb and to contain various exudates discharged from the body.

“Activated” or “pre-activated” refer to a process of mechanically deforming a material in order to increase the extensibility of at least a portion of the material. A material may be activated or pre-activated by, for example, incrementally stretching the material in at least one direction.

“Adhesively bonded” or “adhesively laminated” refer to a laminate wherein an adhesive is used to bond an elastomeric material to at least one cover layer.

“Attached” refers to elements being connected or united by fastening, adhering, bonding, or by any other method suitable for connecting the elements together and to their constituent materials. Many suitable methods for attaching elements together are well-known, including adhesive bonding, pressure bonding, thermal bonding, ultrasonic bonding, mechanical fastening, etc. Such attachment methods may be used to attach elements together over a particular area either continuously or intermittently.

“Diaper” refers to an absorbent article generally worn by infants and incontinent persons about the lower torso and having the general form of a sheet, different portions of which are fastened together to encircle the waist and the legs of the wearer.

“Disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

“Disposed” is used to mean that an element(s) is formed (joined and positioned) in a particular place or position as a unitary structure with other elements or as a separate element joined to another element.

“Extensible” refers to the property of a material, wherein: when a biasing force is applied to the material, the material can be extended to an elongated length of at least 115% of its original relaxed length (i.e., can extend 15%), without a rupture or breakage that renders the material unusable for its intended purpose. A material that does not meet this definition is considered inextensible. In some embodiments, an extensible material may be able to be extended to an elongated length of 125% or more of its original relaxed length without rupture or breakage that renders the material unusable for its intended purpose. An extensible material may or may not exhibit recovery after application of a biasing force.

Throughout the present disclosure, an extensible material is considered to be “elastically extensible” if, when a biasing force is applied to the material, the material can be extended to an elongated length of at least 115% of its original relaxed length (i.e., can extend 15%), without rupture or breakage which renders the material unusable for its intended purpose, and when the force is removed from the material, the material recovers at least 40% of its elongation. In various examples, when the force is removed from an elastically extensible material, the material may recover at least 60%, or at least 80%, of its elongation.

An “elastic,” “elastomer” or “elastomeric” refers to materials exhibiting elastic properties, which include any material that upon application of a force to its relaxed, initial length can stretch or elongate to an elongated length more than 15% greater than its initial length and will substantially recover back to about its initial length upon release of the applied force.

“Interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” include, but are not limited to “inner,” “inside,” “skin-facing,”“skin-side,” “wearer-facing,” or “wearer-side.” Synonyms for “exterior include, but are not limited to “outer,” “outside,” “garment-side,” or “garment-facing.” Also, in the taped diaper context, when the absorbent article is oriented such that its interior faces upward, e.g., when it is laid out in preparation for setting the wearer on top of it, synonyms include “upper” and “lower” and “top” and “bottom”, respectively.

“Joined” refers to configurations whereby an element is directly secured to another element by attaching the element directly to the other element, and configurations whereby an element is indirectly secured to another element by attaching the element to intermediate member(s) which in turn are attached to the other element.

“Pant” or “pants” refers to an absorbent article generally worn by infants and incontinent persons about the lower torso and having the general form of a pair of short pants that can be applied or removed from the wearer without unfastening. A pant may be placed in position on the wearer by inserting the wearer's legs into the leg openings and sliding the pant into position about the wearer's lower torso. While the term “pant” is used herein, pants are also commonly referred to as “closed diapers”, “prefastened diapers”, “pull-on diapers”, “training pants” and “diaper-pants”.

“Refastenable” refers to the property of two elements being capable of releasable attachment, separation, and subsequent releasable reattachment without substantial permanent deformation or rupture.

“Releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

“Strain” or “percent strain” of a material is calculated by subtracting the original length from the stretched length, then dividing the result by the original length and multiplying by 100. The percent strain is described by the equation below:

Percent Strain=% Strain=Strain=100*[(Ls−L₀)/L₀]

where L₀ is the original length of the stretch laminate (or elastomeric film) at the beginning of the stretch step, and Ls is the length of the stretched laminate (or elastomeric film) at the end of the stretch step. A sample stretched from an original length of 10 mm to a length of 30 mm results in a strain of 200%. Strain can be calculated in a length direction, a width direction, or any direction there between.

“Set” or “percent set” of a material is calculated by subtracting an original length from a final length, then dividing the result by the original length and multiplying by 100. The percent set is described by the equation below:

Percent Set=% Set=Set=100*[(L_{f}−L₀)/L₀]

where L₀ is an original length of the stretch laminate (or elastomeric film) at the beginning of the stretch step, and L_{f} is a length of the relaxed stretch laminate (or elastomeric film) after it is relaxed from the stretch step. A sample is stretched from an original length of 10 mm to a length of 30 mm. Upon relaxing (removal of stress), the sample returns to 15 mm. This results in a set of 50%. Set can be calculated in a length direction, a width direction, or any direction there between.

“Wrinkle” refers to a small fold, ridge or crease.

“Align” or “aligned” or “aligning” means to place or to arrange in a straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

“Facing relationship” refers to a relative positioning of materials, such as substrates, in which a surface of one material is oriented toward a surface of another material. For example, when two substrates are stacked on top of each other, they are in a facing relationship. The term does not require or exclude the presence of intervening objects, materials, or layers.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

“Nonwoven” refers herein to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern. It is to be appreciated that nonwovens having various basis weights can be used in accordance with the methods herein. For example, some nonwovens may have a basis weight of at least about 8 gsm, 12 gsm, 16 gsm, 20 gsm, 25 gsm, 30 gsm, 40 gsm, or 65 gsm. Some nonwovens may have basis weight of about 8 gsm to about 65 gsm, or about 10 to about 22 gsm, specifically reciting all 1 gsm increments within the above-recited ranges and all ranges formed therein or thereby. The nonwovens may comprise or be formed of natural fibers, such as cotton, and bio-based fibers, such as bio-PE or bio-PP.

“Pattern” as used herein means a decorative or distinctive design, not necessarily repeating or imitative, including but not limited to the following: clustered, geometric, spotted, helical, swirl, arrayed, textured, spiral, cycle, contoured, laced, tessellated, starburst, lobed, blocks, pleated, concave, convex, braided, tapered, and combinations thereof. In some embodiments, the pattern may comprise one or more repeating design elements.

Lateral or transverse axis of the absorbent article refers to a direction running at a 90 degree angle to the longitudinal direction and includes directions within ±45° of the lateral direction.

Longitudinal axis of the absorbent article refers to a direction running parallel to the maximum linear dimension of the article and includes directions within +45° of the longitudinal direction.

### General Description of an Absorbent Article

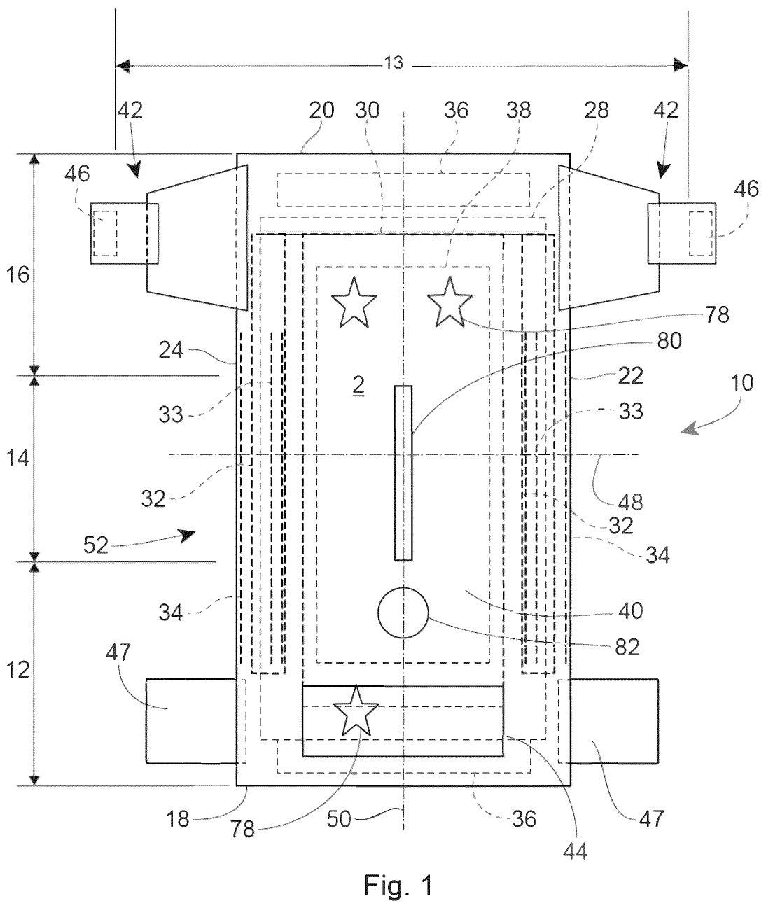

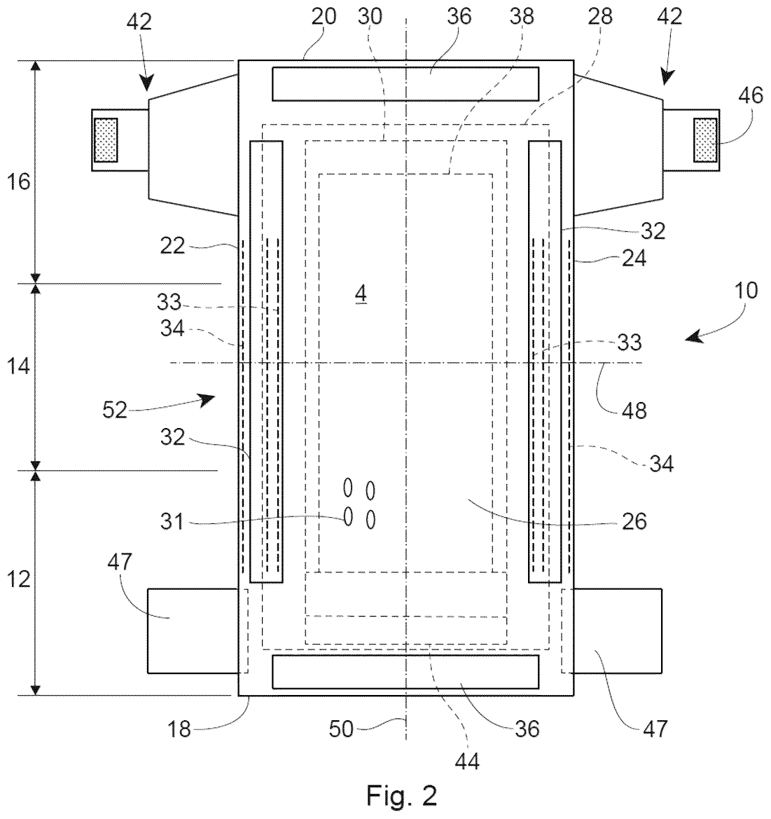

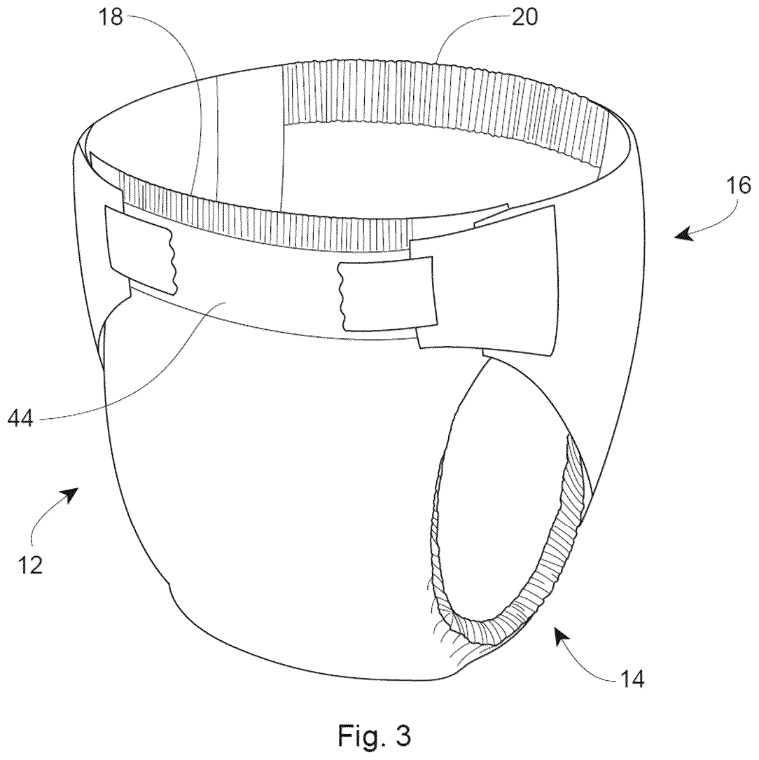

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. A masking layer may be present intermediate the absorbent core and the backsheet film. An outer cover material 40, such as a nonwoven material, may cover a garment-facing side of the backsheet 28. The absorbent article 10 may comprise back ears 42 in the back waist region 16. The back cars 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front cars 47 in the front waist region 12. The absorbent article 10 may have a central lateral (or transverse) axis 48 and a central longitudinal axis 50. The central lateral axis 48 extends perpendicular to the central longitudinal axis 50. The absorbent article may comprise a secondary fastening system in addition to the primary fastening systems in a typed diaper context.

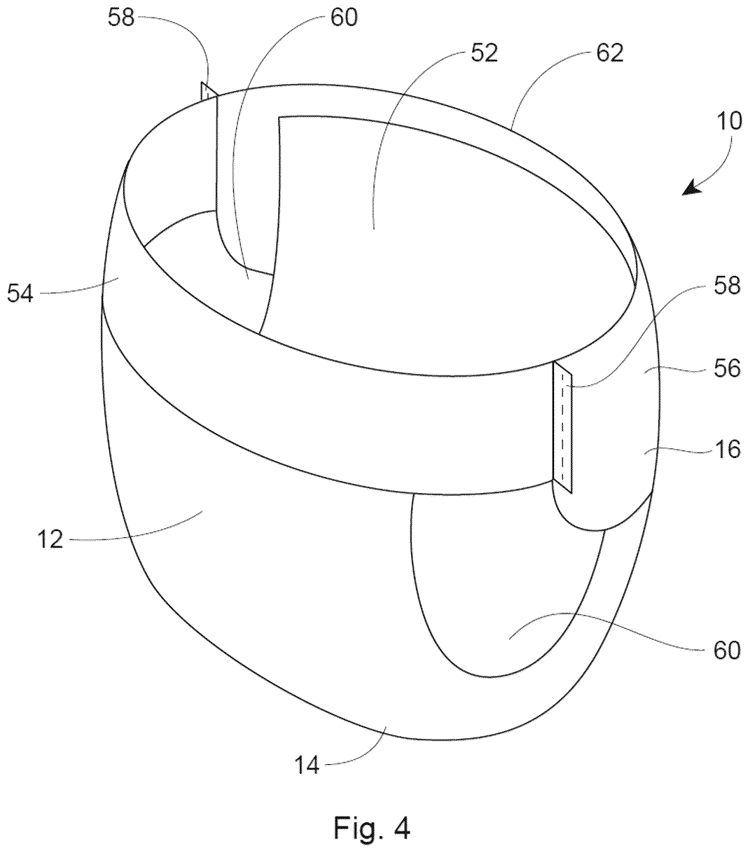

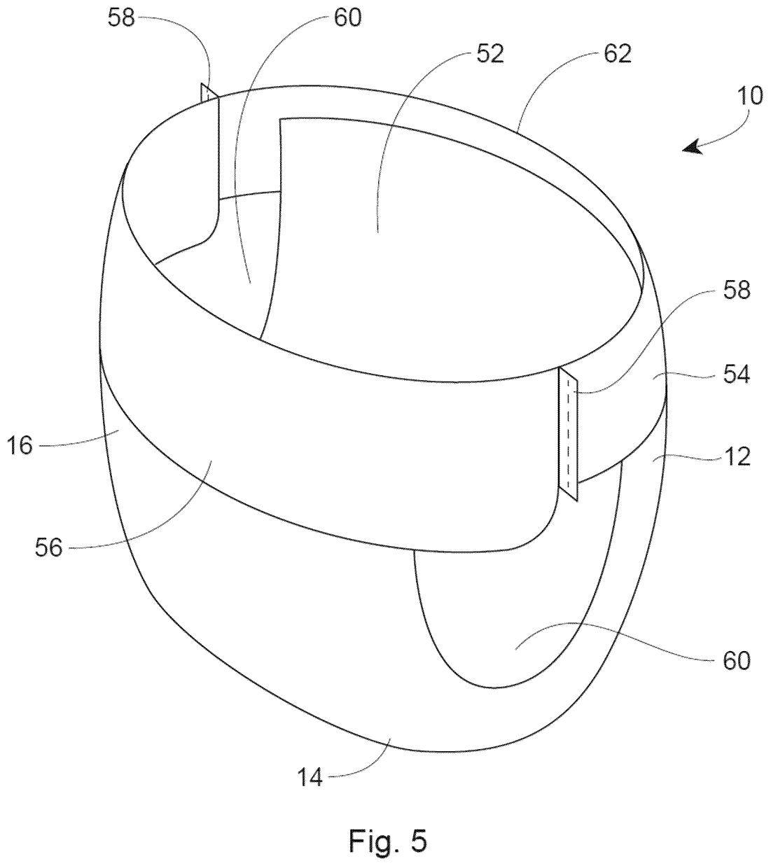

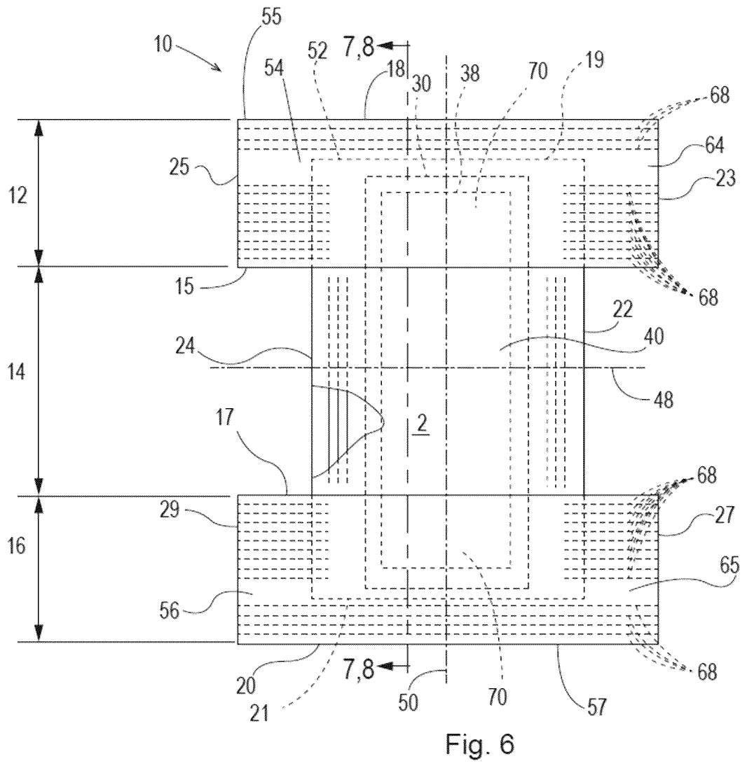

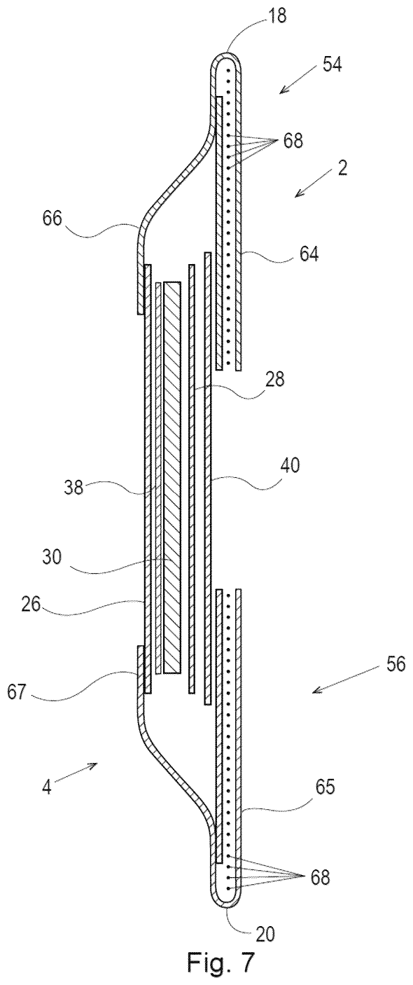

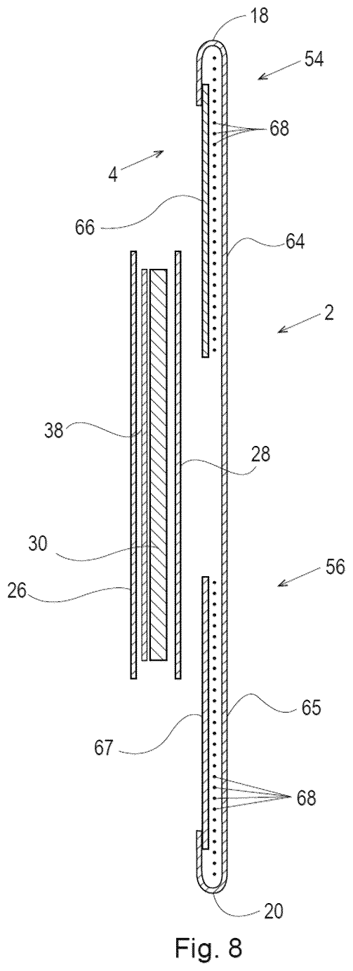

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce clastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds, thermoplastic bonds, or ultrasonic bonds. Various suitable belt layer configurations can be found in U.S. Pat. Appl. Pub. No. 2013/0211363. Any of the belts 54 and 56 may comprise a stretch laminate as described hereinafter.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. The topsheet may be formed of one or more layers of equal or unequal size or area. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or bio-sourced fibers, bio-polymers, PIR polymers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet. The backsheet may be printed with inks for graphics. The backsheet may also be printed with a wetness indicator, such as through the use of one or more hydrochromic inks.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material.

### Absorbent Core

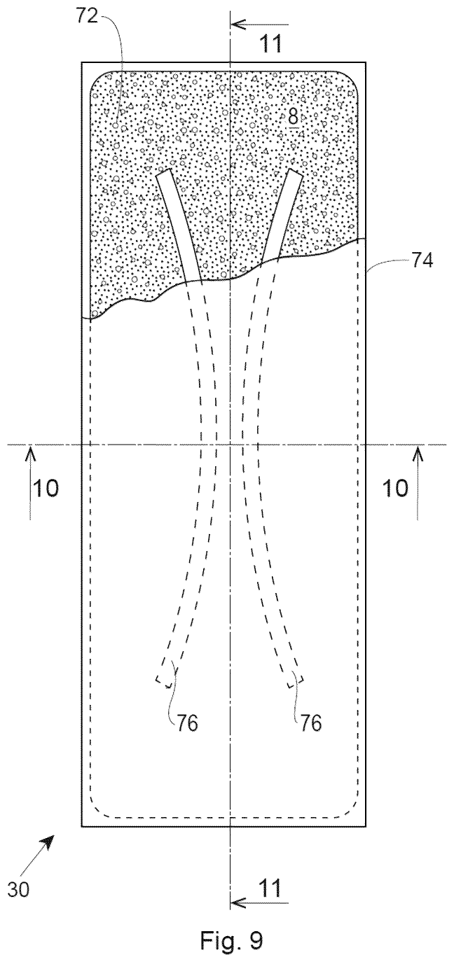

As used herein, the term “absorbent core” 30 refers to a component of the absorbent article 10 disposed in the article for absorbing and containing liquid such as urine received by the absorbent article. The absorbent core thus typically has a high absorbent capacity. An example absorbent core 30 is schematically shown in FIGS. 9-11. The absorbent core comprises an absorbent material 72, that is typically enclosed within or sandwiched between a core bag 74.

The core wrap may be a single material that is folded and attached to itself, or it may comprise a separate top layer and bottom layer that may be bonded or otherwise joined together. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region or towards the front region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. According to various embodiments, the absorbent material may comprise at least 80% superabsorbent polymers by weight of the absorbent material. The absorbent material may also be free of cellulose fibers, as is known in so-called airfelt-free cores, where the absorbent material consists, or consists essentially, of SAP. The absorbent material may also be a high internal phase emulsion foam

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. Appl. Pub. No. 2006/024433 (Blessing), U.S. Pat. Appl. Pub. No. 2008/0312617 and U.S. Pat. Appl. Pub. No. 2010/0051166A1 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure.

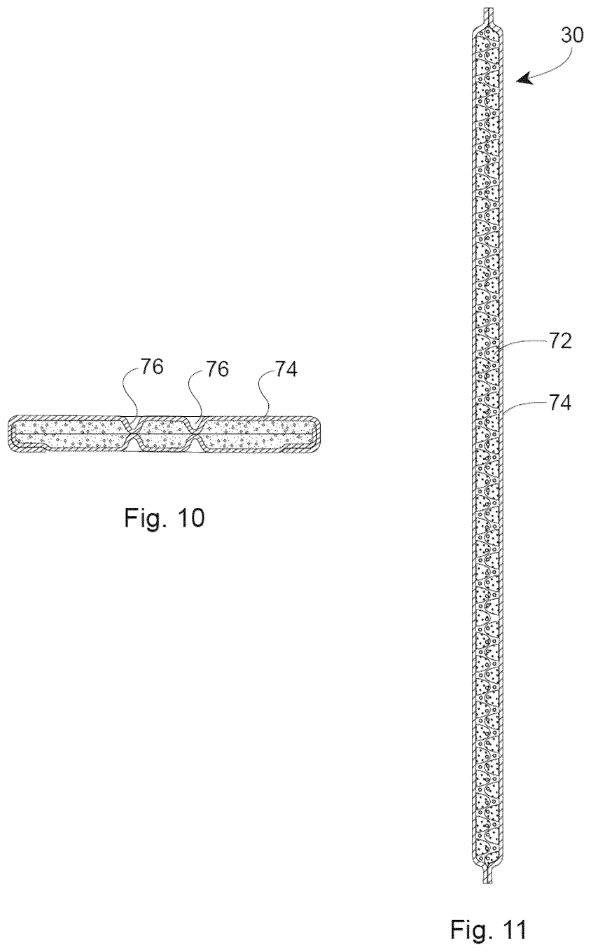

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/312,622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in WO2012/170778 (Rosati et al.) and US2012/0312491 (Jackels). Channels may of course also be formed in absorbent cores comprising a mix of cellulose fibers and SAP particles. These channels may embody any suitable shapes and any suitable number of channels may be provided. For example, the channel(s) may be “T” or “U” or “X” or “8” (figure-eight) shaped channels. The channels may be continuous or intermittent/discontinuous. In other instances, the absorbent core may be embossed to create the impression of channels. It is also to be appreciated that the channels may include both embossed channels and absorbent material-free junction areas, as previously discussed. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., clastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4 or between the garment-facing surface and the wearer-facing surface. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The waistband may comprise clastic strands, elastic films, or combinations thereof. The waistband may be a stretch laminate within the scope of the present disclosure and may be ultrasonically bonded.

### Acquisition Materials

Referring to FIGS. 1, 2, 7, and 8, an acquisition layer comprising one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may include a landing zone area 44. The landing zone area 44 may be formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa. The landing zone may comprise a nonwoven with enough fiber loops to enable adequate fastening.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78. The wetness indicators may be slot coated, gravure printed or digitally printed onto a carrier substrate. The indication action, usually a color change, may be pH-sensitive (blue to green to yellow, blue to yellow, other) or may be thermochromic (temp-sensitive).

### Front and Back Ears

Referring to FIGS. 1 and 2, as referenced above, the absorbent article 10 may have front and/or back cars 47, 42 in a taped diaper context. Only one set of cars may be required in most taped diapers. The single set of cars may comprise fasteners 46 configured to engage the landing zone or landing zone area 44. If two sets of cars are provided, in most instances, only one set of the cars may have fasteners 46, with the other set being free of fasteners. The cars, or portions thereof, may be elastic or may have elastic panels. In an example, an elastic film or elastic strands may be positioned intermediate a first nonwoven material and a second nonwoven material. The elastic film may or may not be apertured. The cars may be shaped. The cars may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. Additionally or alternatively, any of the cars 42, 47 may comprise a stretch laminate as described hereinafter.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g., color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Stretch Laminate

Various elements, particularly elastic components, of the absorbent articles 10 described herein may include a stretch laminate. For example, any of the elastic side members, the belts 54 and 56 and/or any of the cars 42, 47 may comprise a stretch laminate as described hereinafter. The waistbands may also include a stretch laminate. Such laminates may include an elastomeric layer that provides extensibility to the laminate and one or more outer layers that is less stretchable but suitable for providing durability and desirable tactile properties. In this way, the laminate permits a component of an absorbent article to closely and comfortably contact the wearer while providing desirable exterior qualities.

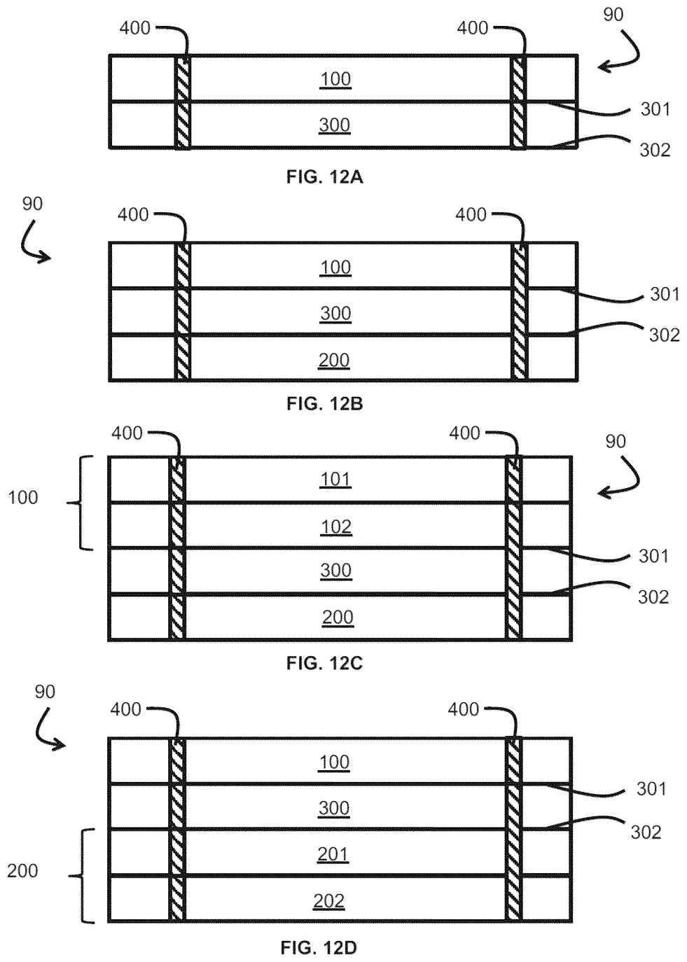

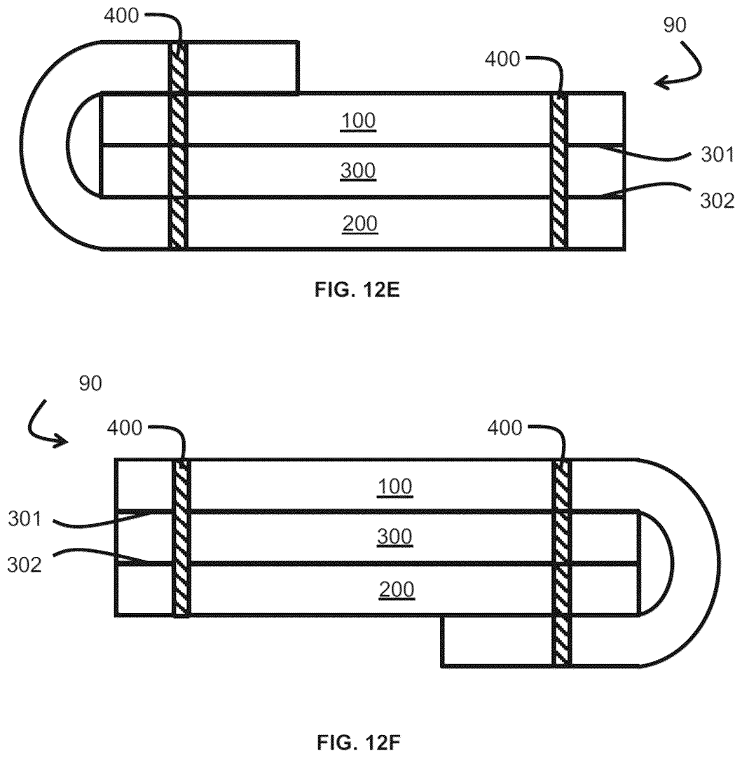

FIGS. 12A-F are a cross-sectional views of various stretch laminates 90. As shown in FIG. 12A, a stretch laminate 90 may comprise a first cover layer 100 and an elastomeric film layer 300 joined via one or more ultrasonic bonds 400. It is also to be appreciated that the layers of the stretch laminate may be bonded using a combination of mechanical bonds, such as thermal bonds, pressure bonds, and ultrasonic bonds. The elastomeric film layer 300 may have one or more skins, such as a first skin 301 providing first surface and a second skin 302 providing second surface. As shown in FIG. 12B, a stretch laminate 90 may comprise a first cover layer 100 and a second cover layer 200 with an elastomeric film layer 300 sandwiched therebetween in a facing relationship to both the first cover layer 100 and the second cover layer 200. All three layers may be joined via one or more ultrasonic bonds 400. As shown in FIGS. 12C and 12D, all or a portion of the first cover layer 100 may comprise one or more layers, such as a first layer 101 and second layer 102, which may have the same composition or different compositions. Similarly, all or a portion of the second cover layer 200 may comprise one or more layers, such as a first layer 201 and second layer 202, which may have the same composition or different compositions. As shown in FIGS. 12E and 12F, a portion of the first cover layer 100 or the second cover layer 200 may be folded over to provide a multi-layered structure on all or a portion of the opposite side of stretch laminate 90.

Elastomeric film layer 300 of stretch laminate 90 may include a single layer or multiple layers of one or more materials that are elastically extensible. The elastically extensible material(s) may be between about 10 μm and about 100 μm, or between about 20 μm and about 60 μm, or between about 30 μm and about 50 μm, or in some embodiments, about 40 μm, in thickness. The elastically extensible material(s) may comprise an elastomeric polyolefin, and in some embodiments, a polyolefin (POE) blown film.

The elastically extensible material includes one or more elastomeric materials which provide elasticity to at least a portion of the layer. Nonlimiting examples of elastomeric materials include film (e.g., polyurethane films, films derived from rubber and/or other polymeric materials), an elastomeric coating applied to another substrate (e.g., a hot melt elastomer, an elastomeric adhesive, printed elastomer, or elastomer co-extruded to another substrate), elastomeric nonwovens, scrims, and the like. Elastomeric materials can be formed from elastomeric polymers including polymers having styrene derivatives (e.g., styrenic block copolymer materials), polyesters, polyurethanes, polyether amides, polyolefins, combinations thereof or any suitable known elastomers including but not limited to co-extruded VISTAMAXX®. Exemplary elastomers and/or elastomeric materials are disclosed in U.S. Pat. Nos. 8,618,350; 6,410,129; 7,819,853; 8,795,809; 7,806,883; 6,677,258 and U.S. Pat. Pub. No. 2009/0258210. Commercially available elastomeric materials include KRATON (styrenic block copolymer; available from the Kraton Chemical Company, Houston, TX), SEPTON (styrenic block copolymer; available from Kuraray America, Inc., New York, NY), VECTOR (styrenic block copolymer; available from TSRC Dexco Chemical Company, Houston, TX), ESTANE (polyurethane; available from Lubrizol, Inc., Ohio), PEBAX (polyether block amide; available from Arkema Chemicals, Philadelphia, PA), HYTREL (polyester; available from DuPont, Wilmington, DE), VISTAMAXX (homopolyolefins and random copolymers, and blends of random copolymers, available from EXXON Mobile, Spring, TX), VERSIFY (homopolyolefins and random copolymers, and blends of random copolymers, available from Dow Chemical Company, Midland, Michigan), and INFUSE (Block copolymer available from Dow Chemical Company). The elastically extensible material may comprise modifying resins.

The elastically extensible material may comprise a variety of additives. Suitable additives including, but not limited to, stabilizers, antioxidants, and bacteriostats may be employed to prevent thermal, oxidative, and bio-chemical degradation of the elastically extensible material. Additives may account for about 0.01% to about 60% of the total weight of the elastically extensible material. In other embodiments, the composition comprises from about 0.01% to about 25%. In other suitable embodiments, the elastically extensible material comprises from about 0.01% to about 10% by weight, of additives.

The elastically extensible material may comprise various stabilizers and antioxidants that are well known in the art and include high molecular weight hindered phenols (i.e., phenolic compounds with sterically bulky radicals in proximity to the hydroxyl group), multifunctional phenols (i.e., phenolic compounds with sulfur and phosphorous containing groups), phosphates such as tris-(p-nonylphenyl)-phosphite, hindered amines, and combinations thereof. Proprietary commercial stabilizers and/or antioxidants are available under a number of trade names including a variety of Wingstay®, Tinuvin® and Irganox® products.

The elastically extensible material may comprise various bacteriostats that are known in the art. Examples of suitable bacteriostats include benzoates, phenols, aldehydes, halogen containing compounds, nitrogen compounds, and metal-containing compounds such as mercurials, zinc compounds and tin compounds. A representative example is available under the trade designation Irgasan Pa. from Ciba Specialty Chemical Corporation of Tarrytown, N.Y.

The elastically extensible material may comprise viscosity modifiers, processing aids, slip agents or anti-block agents. Processing aids include processing oils, which are well known in the art and include synthetic and natural oils, naphthenic oils, paraffinic oils, olefin oligomers and low molecular weight polymers, vegetable oils, animal oils, and derivatives of such including hydrogenated versions. Processing oils also may incorporate combinations of such oils. Mineral oil may be used as a processing oil. Viscosity modifiers are also well known in the art. For example, petroleum derived waxes can be used to reduce the viscosity of the slow recovery elastomer in thermal processing. Suitable waxes include low number-average molecular weight (e.g., 0.6-6.0 kilo Daltons) polyethylene; petroleum waxes such as paraffin wax and microcrystalline wax; atactic polypropylene; synthetic waxes made by polymerizing carbon monoxide and hydrogen such as Fischer-Tropsch wax; and polyolefin waxes.

The desirability and noticeability of many of stretch laminate features have been tested with consumers. Nonwoven choice can influence the appearance of a bond pattern as well as change the tactile softness of the laminate. Changes in ultrasonic and nonwoven bond pattern can change the texture of the stretch laminate, both tactically and visually. In short, varying these parameters in a laminate can make clear differentiation that is consumer noticeable and appreciated. Some further variations are detailed hereinafter.

The attachment of the layers of the stretch laminate are discussed herein as having ultrasonic bonds. However, it is to be appreciated that other types of mechanical bonding may be used in combination with the ultrasonic bonds to from the stretch laminate and the following disclosure is applicable to this combination of bonds. For example, a stretch laminate may include ultrasonic and thermal bonds or ultrasonic and pressure bonds.

The area of the laminate including the bond pattern may have a Load Force at 50% of about 0.3 N/in or greater, or about 0.4 N/in or greater, or from about 0.45 to about 2 N/in, reciting for said range every 0.05 N/in increment therein, according to the Hysteresis Test Method herein.

The area of the laminate including the bond pattern may have an Unload Force at 50% of about 0.2 N/in or greater, or about 0.3 N/in or greater, or from about 0.35 to about 1 N/in, reciting for said range every 0.05 N/in increment therein, according to the Hysteresis Test Method herein.

The area of the laminate including the bond pattern may have a Smax of about 50% or greater, or about 75% or greater, or from about 100% to about 300% or from about 100% to about 500%, reciting for said range every 1% increment therein, according to the Hysteresis Test Method herein.

### Skin

Exemplary elastomeric film layers 300 that are useful in the stretch laminates 90 detailed herein (i.e., an elastically extensible material with at least one skin disposed on the surface of the elastically extensible material) include M18-1117 and M18-1361 elastomeric films commercially available from Clopay Corporation of Cincinnati, Ohio; K11-815 and CEX-826 elastomeric films commercially available from Tredegar Film Products of Richmond, Virginia; and elastomeric films commercially available from Mondi Gronau GmbH of Gronau, Germany. These exemplary elastomeric films may include a single layer of elastically extensible material with a skin 301, 302 disposed on both surfaces of the material. Other elastomeric film layers applicable to the stretch laminates detailed herein need not have a skin 301, 302 on both surfaces of the material and may instead have no skin or a skin on only one surface.

### Nonwoven Material

The first cover layer 100 and the cover layer material 200, as well as any layers 101, 102, 201, 202 that make up either material, may include any suitable nonwoven material or combination of nonwoven materials, including but not limited to, spun only or spun meltblown combinations, such as SM (spunbond meltblown), SMS (spunbond meltblown spunbond), SMMS (spunbond meltblown spunbond) nonwovens, SSMMS (spunbond meltblown spunbound), hydroentangled nonwovens and softbond nonwovens. The nonwoven materials may also include carded nonwovens, such as those specially designed and manufactured to be compatible with an activation (e.g., ring-rolling) process. One exemplary nonwoven material is a carded nonwoven made from a polypropylene homopolymer. The spunbounds may also be specially designed and/or manufactured to be compatible with an activation process. However, it is believed that through the use of the elastomeric film according to the present disclosure, greater flexibility in the design choices may be achieved. For example, spunbounds may be selected for applications where only carded nonwovens were used in the past, or thinner elastomeric films may be used with the carded nonwovens. Other improvements in design flexibility will also be recognized by the skilled practitioner. For example, in some embodiments, the cover layer(s) may be extensible nonwovens and may or may not need to undergo an activation process in order to impart extensibility to the stretch laminate.

The basis weight of the nonwoven material may be less than about 30 gsm. In fact, according to certain embodiments, the basis weight may be less than about 27 gsm. In other embodiments, the basis weight may be less than about 25 gsm. In still other embodiments, the nonwoven material may have a basis weight of less than about 24 gsm. The nonwoven materials may also include additives, such as, for example, CaCO₃. Woven or knitted fabrics may also be used as cover layers 100, 200 in embodiments of the stretch laminates 90 detailed herein.

The ultrasonic bonds 400 preferably eliminate the need for any adhesives, but adhesives may be employed to join the layers 100, 200, 300 of the stretch laminate 90. Adhesives may be selected from any adhesives known to provide suitable attachment between elastomeric film layer 300 and cover layers 100, 200. In some embodiments, the adhesive may be a hot melt adhesive with a basis weight of less than about 15 gsm. According to one embodiment, the adhesive may be H2031 adhesive commercially available from Bostik Inc. of Middleton, Massachusetts. One characteristic of this adhesive is that, at 23° C., this adhesive has significant pressure-sensitive character useful for making a stretch laminate by hand. However, this adhesive is also suitable for use in fabricating stretch laminates from the elastomeric films and cover layers listed above using conventional stretch laminate manufacturing equipment, such equipment being well known in the art.

The nonwoven may include added materials such as inks, color-changing indicators, and skin compositions, such as moisturizers, fragrances, lubricants, anti-bacterial substances, bug repellency substances, and UV-protection substances. The added materials may be disposed on the nonwoven or one or more layers of the laminate. The added materials may be disposed on the surface. For example, inks may be applied to the nonwoven by printing. The ink may provide a visual signal to the user, such as the location of a bonds or the bond pattern or a graphic.

Bond pattern of the laminate may coordinate with other portions of the absorbent article. For example, the bond pattern of the laminate may coordinate with a pattern on the chassis, such as the topsheet and/or the backsheet, the ears, the fasteners, and/or the waist feature. The coordinating pattern may be a pattern formed by mechanically changing the structure of the material or adding materials to from a pattern, such as by printing.

### Pre-Activation

Elastomeric film layer 300 may be mechanically pre-activated before attachment to at least one cover layer 100, 200. For example, elastomeric film layer 300 may be pre-activated by being stretched transversely to its web direction by more than 50% (i.e., strain >50%). In some embodiments, an expansion by about 100% to about 500% occurs in relation to the starting width of the elastomeric film layer 300. In alternate embodiments, elastomeric film layer 300 may be stretched in the web direction, stretched a direction other than the web direction or transverse to the web direction, or a combination of directions. The term “stretching” is to point to the fact that the expansion of elastomeric film layer 300 is not completely reversible and that a non-clastic fraction results in the film having a larger width following pre-activation (i.e., the elastomeric film does not have 100% recovery, and therefore has a percent set value). After expansion, elastomeric film layer 300 retracts and has a width that may be larger by about 10% to about 30% in relation to a starting width of the elastomeric film layer. In other words, after the pre-activation expansion and retraction detailed below, elastomeric film layer 300 may exhibit a set of about 10% to about 30%.

According to various embodiments in which elastomeric film layer 300 includes both an elastically extensible material and at least one skin disposed on the elastically extensible material, the pre-activation process may physically alter these materials differently, for example, because these materials have different elasticity and recovery properties. During pre-activation, the skin 301 and/or 302 and the elastically extensible material are similarly stretched (i.e., put under similar strain). However, after stretching, the skin and the elastically extensible material will retract and recover differently (i.e., have different set values). In comparison with the elastically extensible material, the skin is less elastic and therefore will have less recovery after stretching, a.k.a., a higher set value. The skin is also much thinner than the elastically extensible material, so when the thicker elastically extensible material retracts and recovers after pre-activation stretching, it will force the attached skin to retract with it. But because the skin cannot recover as much as the elastically extensible material, the skin buckles and wrinkles. Accordingly, the cross-sectional profile and the top view appearance of elastomeric film layer 300 are modified after a pre-activation process.

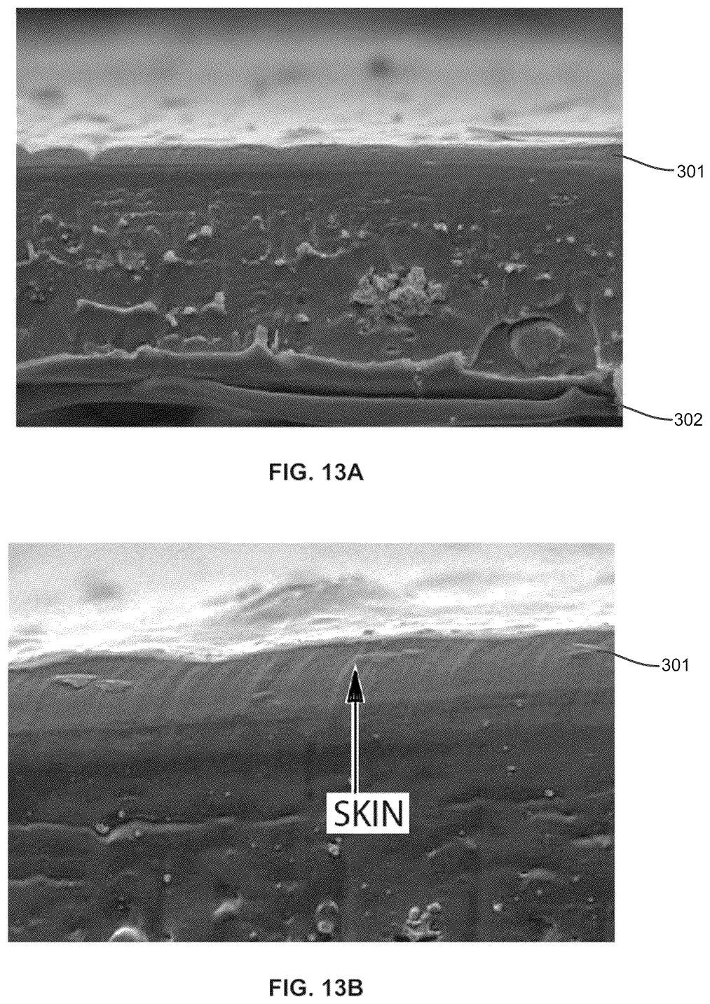

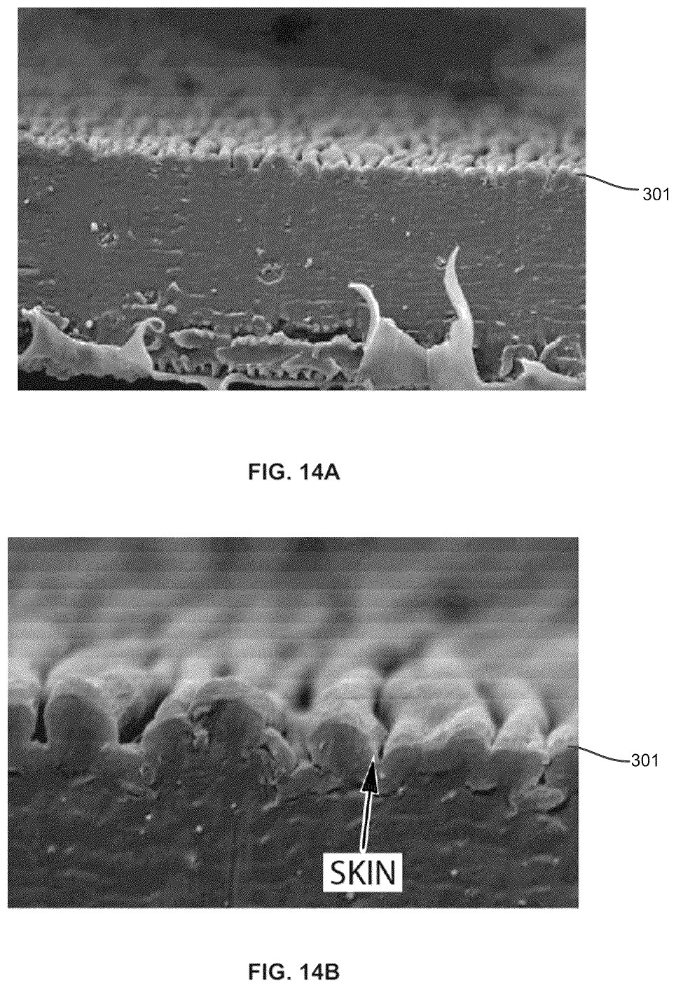

FIGS. 13A, 13B, 14A, and 14B are SEM photomicrographs of magnified cross-sections of elastomeric films. These SEM photomicrographs, as well as the other SEM photomicrographs included herein, were taken with a scanning electron microscope (Hitachi Model 3500). The information to calculate specific magnifications and distances is included in each individual SEM photomicrograph along the bottom of the frame. FIG. 13A is a SEM photomicrograph taken at approximately 900× magnification showing a cross-sectional view of a portion of an elastomeric film that has not been pre-activated. The skins 301, 302 are the thin strips of contrasting material at the top and the bottom of the cross-section, with the thicker elastically extensible material between the skins 301, 302. The skin 301 at the top of the cross-section is easier to discern due to the cross-section being cut cleaner in that region. Without pre-activation, the skins 301, 302, and thus the outer surfaces of the elastomeric film, are substantially smooth in a cross-sectional view. FIG. 13B is a higher magnification image (approx. 3500× magnification) of the skin 301 at the top of cross-section shown in the SEM photomicrograph of FIG. 13A.

FIG. 14A is a SEM photomicrograph taken at approximately 900× magnification showing a cross-sectional view of a portion of an elastomeric film that has been pre-activated. Again, the skins 301 are the thin strips of contrasting material at the top and the bottom of the cross-section, with the thicker elastically extensible material between the skins. With pre-activation, the skins 301, and thus the outer surfaces of the elastomeric film, are wrinkled in a cross sectional view. FIG. 14B is a higher magnification image (approx. 3500× magnification) of the skin at the top off the cross-section shown in the SEM photomicrograph of FIG. 14A.

FIGS. 14A and 14B show that after pre-activation, the skin 301 of elastomeric film layer 300 includes a plurality of wrinkles having hills and furrows. For example, as shown in the non-limiting sample photographed in FIG. 14B, there are approximately six hills and six furrows of varying size within the pictured approximately 35 μm of length taken along the cross-sectional profile of the pre-activated elastomeric film. This is in comparison to FIG. 13B, in which there are no hills and no furrows within the pictured approximately 35 μm of length taken along the cross-sectional profile of an elastomeric film that was not pre-activated. However, as visible on the top surface of the elastomeric film shown in FIG. 14B, one or more random hills and/or furrows may be present within a particular length of cross-sectional profile of an elastomeric film that was not pre-activated. These random hills and/or furrows are due to irregularities in the surface of the elastomeric film. Such random hills and/or furrows should not be confused with the hills and furrows of the plurality of wrinkles that are intentionally formed in an elastomeric film through a mechanical pre-activation process.

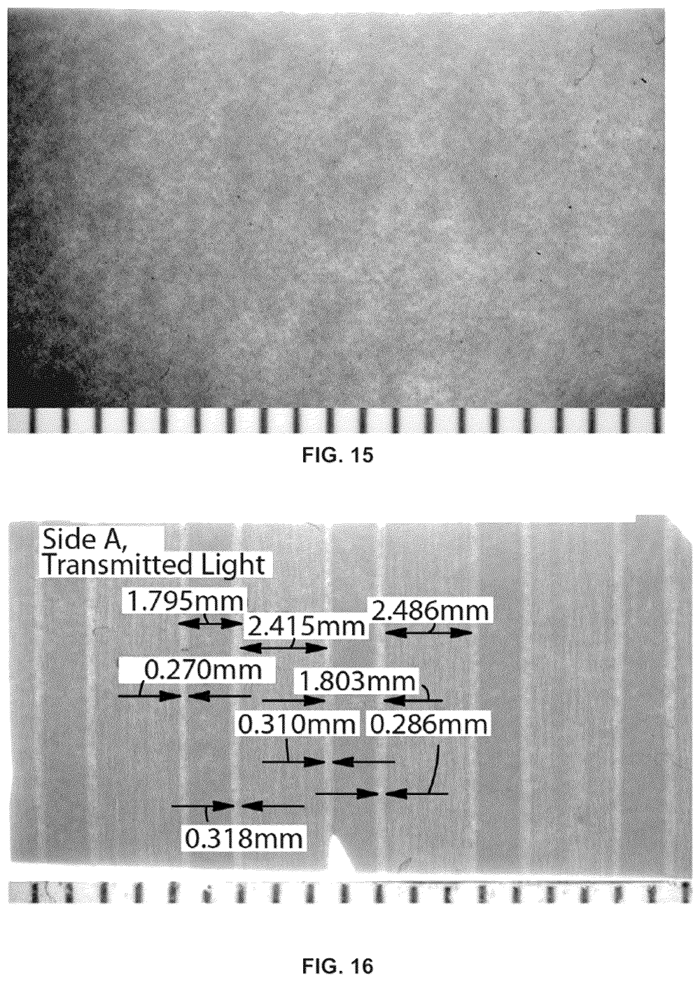

FIGS. 15 and 16 are transmitted light photomicrographs of magnified top views of elastomeric films. The transmitted light photomicrographs were taken in color using a Nikon SMZ 1500 Stereo Light Microscope equipped with an Evolution Mp5C Digital camera with white light shining underneath the elastomeric film samples. The blue scale marks at the bottoms of FIGS. 15 and 16 are in millimeters. This scale can be used to calculate specific magnifications and distances in the transmitted light photomicrographs. FIG. 15 is a transmitted light photomicrograph showing a top view of a portion of an elastomeric film that has not been pre-activated. Without pre-activation, the viewable outer surface of the elastomeric film (i.e., the top view of the skin), has no discernible stripes and is uniform in appearance. FIG. 16 is a transmitted light photomicrograph showing a top view of a portion of an elastomeric film that has been pre-activated. With pre-activation, the top view of the skin includes a plurality of stripes in varying thicknesses that relate to the size and pitch of the intermeshing discs of the mechanical pre-activation means. The stripes, referred to herein as activation stripes, indicate zones in the pre-activated elastomeric film in which there was a particular range of stretching during the pre-activation process. For example, as shown in non-limiting sample photographed in FIG. 16, there are medium thickness darker blue stripes indicative of a heavier intensity skin wrinkling, large thickness light blue stripes indicative of medium intensity skin wrinkling, and thin white stripes indicative of lower intensity skin wrinkling.

In addition, after preactivation, but before utilizing elastomeric film layer 300 in the fabrication of stretch laminate 90, the film layer 300 may optionally be printed with an image or motif that may show through the cover layers 100, 200 of the stretch laminate 90. The ink or other pigment utilized in printing will be deposited on the hills and into the furrows of the wrinkles of the pre-activated elastomeric film. Ink deposited onto the textured surface of a pre-activated elastomeric film allows for more contact surface area between the elastomeric film and the ink. Accordingly, when printing on a pre-activated elastomeric film, there is an image that is more strongly set on the film when compared to an image printed on the much smoother surface of an elastomeric film that has not been pre-activated.

Moreover, when stretch laminate 90 includes a pre-activated (and subsequently printed) elastomeric film that is mechanically, a non-distorted printed image on the film is evenly and reversibly stretched along with it. This is because before the image was printed on the pre-activated elastomeric film, a significant portion, or the entire, non-elastic fraction of elastomeric film 90 has already been removed in the pre-activation process. In other words, the set had been removed from elastomeric film layer 300 before printing. Therefore, the printed image will not substantially distort further with the later activation of stretch laminate 90, or in additional stretching of the laminate by a user. In contrast, if an image or motif were printed on an elastomeric film that was not pre-activated, and that printed film was then used in fabricating a stretch laminate, and then the stretch laminate was mechanically activated, the desired image would be distorted in the final activated stretch laminate. This is because the set of the elastomeric film was not removed prior to the printing process, and such set would be removed from the elastomeric film in the mechanical activation of the fabricated stretch laminate, thus distorting the original printed image. Likewise, if an elastomeric film is printed and then subsequently preactivated, the set of the elastomeric film will not be removed prior to the printing process, and such set would be removed from the elastomeric film in the pre-activation process, thus distorting the original printed image.

In an embodiment, a pre-activated elastomeric film may be stretched again during the printing of the film. The printed film is then relaxed and used in fabrication and activation of the stretch laminate. The resulting activated stretch laminate has an image or motif that is aesthetically pleasant when the stretch laminate is in a stretched condition during use (e.g., when a user stretches the stretch laminate in application or removal of an absorbent article).

In embodiments of stretch laminates that include an elastomeric film that is pre-activated and subsequently printed, the ink or other pigment utilized in printing will be deposited on the hills and into the furrows of the wrinkles of the film. As detailed above, ink deposited onto the textured surface of a pre-activated elastomeric film will more strongly set on the film due to the additional contact surface area between the elastomeric film and the ink (in comparison to ink disposed on an elastomeric film that has not been pre-activated).

In addition, pre-activating an elastomeric film also lowers the force needed to later stretch the film (versus a non-activated film). This helps the later mechanical activation of the stretch laminate because the load required to activate a stretch laminate that is made with pre-activated film will be lower (versus a non-activated film).

### Stretch Laminate Fabrication Method

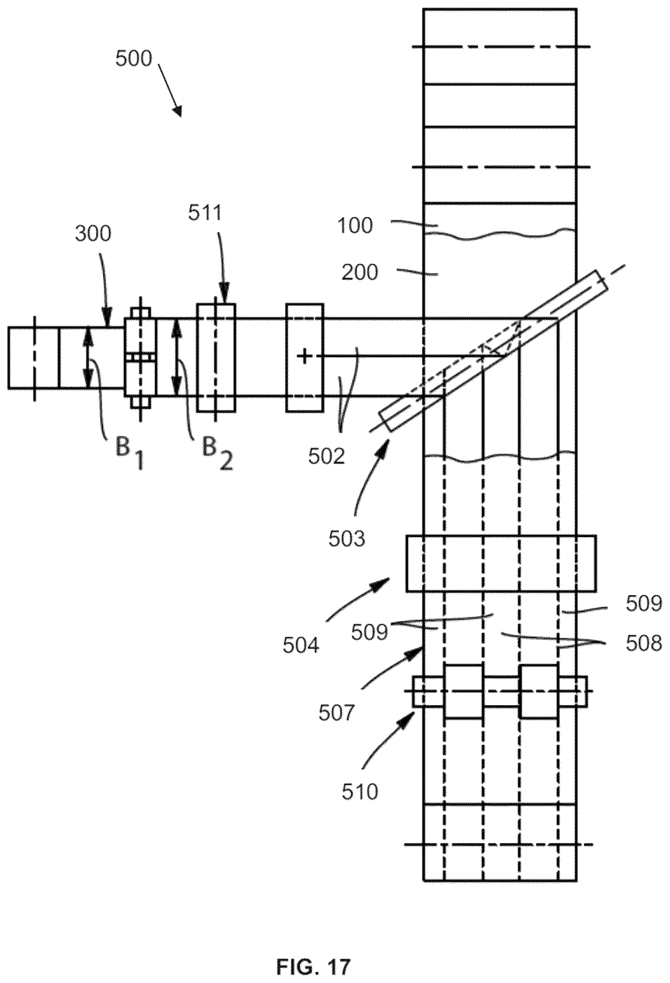

The schematic illustration of FIG. 17 details an exemplary embodiment of a method 500 for fabricating the stretch laminates 90 detailed herein. The method 500 includes providing and pre-activating an elastomeric film 300. Elastomeric film 300 is mechanically pre-activated by stretching the film transverse to its web direction by more than 50%. In some embodiments, an expansion by about 100% to about 500% occurs in relation to the starting width of elastomeric film 300. The term “stretching” is to point to the fact that the expansion of elastomeric film 300 is not completely reversible and that a non-elastic fraction results in the film having a larger width following retraction (i.e., reverse expansion). After expansion, elastomeric film 300 retracts and has a width B₂ that is larger by about 10% to about 30% in relation to a starting width B₁ of the film. Accordingly, elastomeric film 300 has a set of about 10% to about 30% resulting from the pre-activation process.

For the pre-activation process, elastomeric film 300 may be guided through a system of intermeshing profile rollers, each roller including disk packets having a plurality of intermeshing disks that are situated on an axis (i.e., a ring rolling process). Elastomeric film 300 is transversely stretched by the intermeshing disk packets. The stretching may be uniform or varied over the width of the film. The pre-activation process can be carried out at varying pitch and or varying depths of engagement. The pre-activation process can also be carried out in machine direction, or in any other direction. The pre-activation of elastomeric film has a positive effect on the stretching force profile and helps allow for an easy stretching action of the fabricated stretch laminate over a large expansion area. Further, the recovery of the stretch laminate can also be improved by pre-activating the elastomeric film 300. The recovery is the ability of a stretch laminate to return to original size after it has been stretched to its expansion limit. The increased recovery of elastomeric film 300 after the pre-activation process is due to the removal of an amount of set from the film.

After preactivation, but before cutting elastomeric film 300 into film strips 502, the film may optionally be printed in a printing station 511 with an image or motif that may show through the cover layers of the stretch laminate. Any known continuous printing methods can be used for printing the elastomeric film 300. Non-limiting exemplary printing methods include digital printing, inkjet printing, and rotary printing methods, in particular, flexography. As a non-limiting example, the printed image or motif can be a striped motif made of parallel colored stripes that extend in the web's longitudinal direction of elastomeric film 300.

The pre-activated, and optionally printed, film may then be cut into film strips 502. The film strips 502 are guided across redirecting means 503 and supplied to laminating means 504 as parallel strips. Film strips 502 are then laminated in laminating means 504 between cover layers 100, 200, which are supplied above and below the film strips. Film strips 502 and cover layers 100, 200 may be glued together or connected to each other by thermal means, such as ultrasonic bonding, to form composite material 507 (i.e., an embodiment of the stretch laminate materials detailed herein). As illustrated in FIG. 17, film strips 502 are laminated at a distance relative to each other between cover layers 100, 200. Cover layers 100, 200 may, therefore, be directly connected to each other in the regions between film strips 502. Accordingly, clastic regions 508, as well as non-clastic regions 509, may optionally be created in composite material 507. The distance between film strips 502 can be adjusted by positioning the redirecting means. It is also contemplated that reinforcement strips may be laminated between film strips 502 to reinforce non-clastic regions 509 between the film strips.

Composite material 507 is then supplied to an activation means 510 in which the composite material is stretched at portions of elastic regions 508 transversely in relation to the direction of the web. For the stretching, composite material 507 may be guided through a nip between two profile rollers, each roller including at least two disk packets having a plurality of intermeshing disks that are situated on an axis. Composite material 507 is transversely stretched in places by the intermeshing disk packets. The regions in which composite 507 is stretched by the intermeshing disk packets are referred to as stretch zones. In the roller sections between and/or outside the disk packets, the profile rollers form a gap, through which composite 507 is guided though essentially without transverse stretching. The regions in which composite 507 is not stretched by the intermeshing disk packets are referred to as anchoring zones. In the stretch zones, the fibers of cover layers 100, 200 are modified and irreversibly stretched due to fiber tears and rearrangements. Accordingly, the expansion property of composite material 507 is improved in the stretch zones in the cross direction (i.e., transverse in relation to the longitudinal web direction). Following activation, when applying minimal force, composite material 507 is easily expandable in the cross direction to an expansion limit that is preset by the stretching of activation means 510.

When traditional nonwovens are utilized as the cover layers, any pre-activation of elastomeric film 300 cannot replace but can only supplement the mechanical activation of composite material 507. Accordingly, even when elastomeric film 300 is pre-activated, it is still necessary for composite material 507 to be stretched transversely relative to the direction of the web in the regions that are to be rendered elastic via laminated elastomeric film strips (i.e., stretch zones). However, there may be some embodiments of composite material 507 that use extensible nonwovens as the cover layers, and therefore it may not be necessary to activate the composite material.

The laminate may include gathers, wherein one of the layers is strained to a greater degree than a remaining layer during lamination. In this way, the less extensible layer (i.e., the coverstock layer(s)) will form gathers when the laminate is in a relaxed state. In some embodiments, at least a portion of the elastomeric layer is strained while the nonwoven(s) are in a relaxed state during lamination. The elastomeric layer may be stretched in one or more directions. Corrugations then form in the nonwoven layer(s) when the subsequently formed laminate is in a relaxed state. When making laminates that include gathers or gathered laminates, the elastomeric layer is stretched in the stretch direction (i.e., the intended direction of stretch in the final product). The stretch direction may be lateral. In nonlimiting examples, the elastomeric layer is stretched in a direction corresponding with the lateral direction of the article. In other words, when the laminate is joined to the chassis subsequent to lamination, the laminate will be oriented such that the laminate is stretchable in the lateral direction of the article (i.e., the laminate is laterally-extensible).

### Bond Shapes

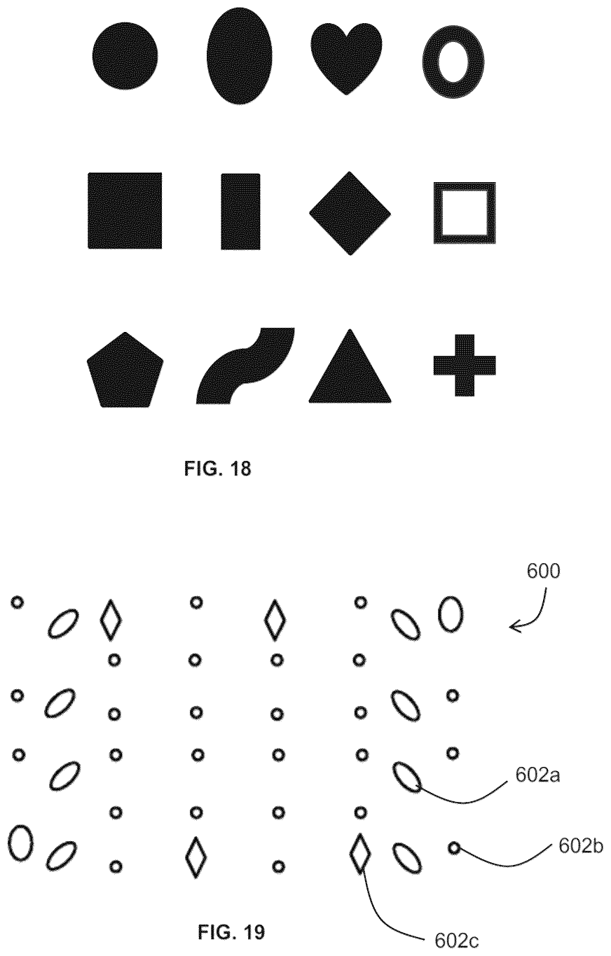

As shown in FIG. 18, individual ultrasonic bonds may be formed in a variety of shapes, including but not limited to curved shapes, straight-sided shapes, and combinations thereof. Examples of curved shapes include but are not limited to circles, ovals, and wavy lines. Examples of straight-sided shapes include but are not limited to triangles, squares, rectangles, diamonds, pentagons, hexagons, octagons, or any n-sided shapes. With respect to n-sided shapes, the shape may have any number of sides, for example, the shape could have from 3 to 12 sides. An example of a shape that is a combination of a curved shape and straight-side shape is a heart. Further, the bond shape may be annular or some other hollow shape, such as illustrated in FIG. 18. Providing various ultrasonic bond shapes may help to add visual appeal and to distinguish between various absorbent articles without compromising bond strength.

### Bond Patterns

Groups of ultrasonic bonds or combinations of ultrasonic bonds and other types of bonds (such as pressure and/or thermal bonds) may be arranged in units and the units may be arranged to form patterns. FIG. 19 is a schematic illustration of a bond pattern 600. The pattern 600 shown in FIG. 19 also comprises a plurality of bond shapes, including an oval bond shape 602a, a circular bond shape 602b, and a diamond bond shape 602c.

“Unit” is a smallest building block of a pattern, whose geometric arrangement defines pattern's characteristic imagery and whose repetition in space is necessary to re-construct the pattern. A pattern may be formed from one or more units. A “repeating unit” is a unit that is substantially the same (i.e., slight variation of dimensions, shape, and/or size) or that is the same and is present multiple times within a pattern; said unit may be rotated, mirrored, or otherwise reoriented. The repeating unit is considered substantially the same if its size and/or shape is within 10% of another repeating unit.

A bond pattern has a bond density. “Bond density” refers to the number of bonds per unit area. FIGS. 20 and 21 are schematic illustrations of stretch laminates 90 having different bond densities, the stretch laminate 90 in FIG. 20 having a lower bond density than the laminate shown in FIG. 21.

Traditional bond patterns consist of a plurality of ultrasonic bonds that are disposed in a grid pattern, as illustrated in FIG. 21, or an off-set grid pattern, as illustrated in FIG. 20. These patterns have been preferred due to the case of manufacturing (allowing for manufacturing at relatively fast speeds while maintaining quality) and the wide applicability to various components of the absorbent article. However, ultrasonic bonds may be used to create various designs in the stretch laminates that communicate to the end user signals of stretch, softness, quality, and the like. Further, more intricate bond patterns have the ability to differentiate products versus competition. With more intricate ultrasonic bond patterns comes relatively more issues with processability and product performance. These more intricate ultrasonic bond patterns generally have individual ultrasonic bonds that are placed more closely together. It has been found that if the bonds are placed in a certain vicinity with respect to one another, the stretch laminate then becomes prone to ruptures when in use, such as tearing in the clastic film. It is believed that the relatively close spacing of certain bonds creates areas of high stress concentration which results in the formation of ruptures in the stretch laminate when the laminate is stretched and held in a stretched state during use. These ruptures may aid in performance of the laminate by making the laminate relatively more breathable.

FIGS. 22A and 22B each illustrate a plan view of an exemplary side member comprising stretch laminates 90 having more than one bond pattern. The exemplary side member may be a first elastic side member 914 or a second clastic side member. As illustrated, the stretch laminate may include a first bond pattern 604 and a second bond pattern 606. The second bond pattern may differ from the first bond pattern in at least one of shape of bonds, number of bonds, shape of repeat unit, bond density, and combinations thereof. The second bond pattern may be positioned outside of the first bond pattern, such that the two patterns may be in non-overlapping relationship. Such as illustrated in FIGS. 22A and 22B, the first bond pattern 602, shown as a plurality of ultrasonic bonds in a grid-like pattern, may be disposed along one or more edges of the laminate. In this way, the first bond pattern 602 may at least partially surround or frame at least a portion of the second bond pattern 604. A dashed line illustrates the imaginary boarder between the first bond pattern 602 and the second bond pattern 604. It is also to be appreciated that in some embodiments, the first bond pattern and the second bond pattern may overlap in a transition zone.

The second bond pattern 606 may include a plurality of bonds positioned in bond site clusters 608. Each bond site cluster includes two or more ultrasonic bonds that are each longitudinally oriented. It has been found that bond site clusters 608 may be used to control rupturing of the stretch laminate to achieve relatively greater breathability of the stretch laminate when in a stretched, in-use state. The bond site cluster create an area of relatively high stress in the laminate when the laminate is extended in a stretch direction. These areas of relatively high stress result in rupturing of the laminate when in use. Ruptures refer to any apertures or tears formed in at least one of the cover layer and the clastic film. However, to continue to perform as intended, such as for use in an absorbent article, the ruptures need to be controlled.

To control these areas of high stress concentrations that result in ruptures in the stretch laminate, it has been found that the longitudinal and transverse spacing between bonds should be controlled. More specifically, the closeness of adjacent bonds depending on their orientation with respect to the longitudinal and transverse direction is controlled to allow for stress concentrations that result in rupturing a predetermined amount. Each bond within the bond site cluster must be a certain distance from each adjacent bond within the bond site cluster and the cluster must be a certain distance from any other adjacent bond site cluster and/or other bond to prevent additional unwanted rupturing. Further, it has been found that bonds within a bond site cluster should be longitudinally oriented bonds with respect to one another to form ruptures. Transversely-oriented bonds are less likely to contribute to rupture in the stretch laminate. The farther a bond is placed away from the 35 degrees from the longitudinal bond axis 618, the more it becomes unlikely that bond will contribute to the rupture of the laminate in the stretch direction. Bonds that oriented along the bond transverse axis 620 are very unlikely to contribute to rupture of the laminate in the stretch direction. Controlling the spacing of adjacent bonds with respect to a primary bond and the dimensions of the bond site clusters allows the stretch laminate, which may be a side panel or an car, to not exhibit a rupture of more than 5 mm according to the Back Ear Hang Test.

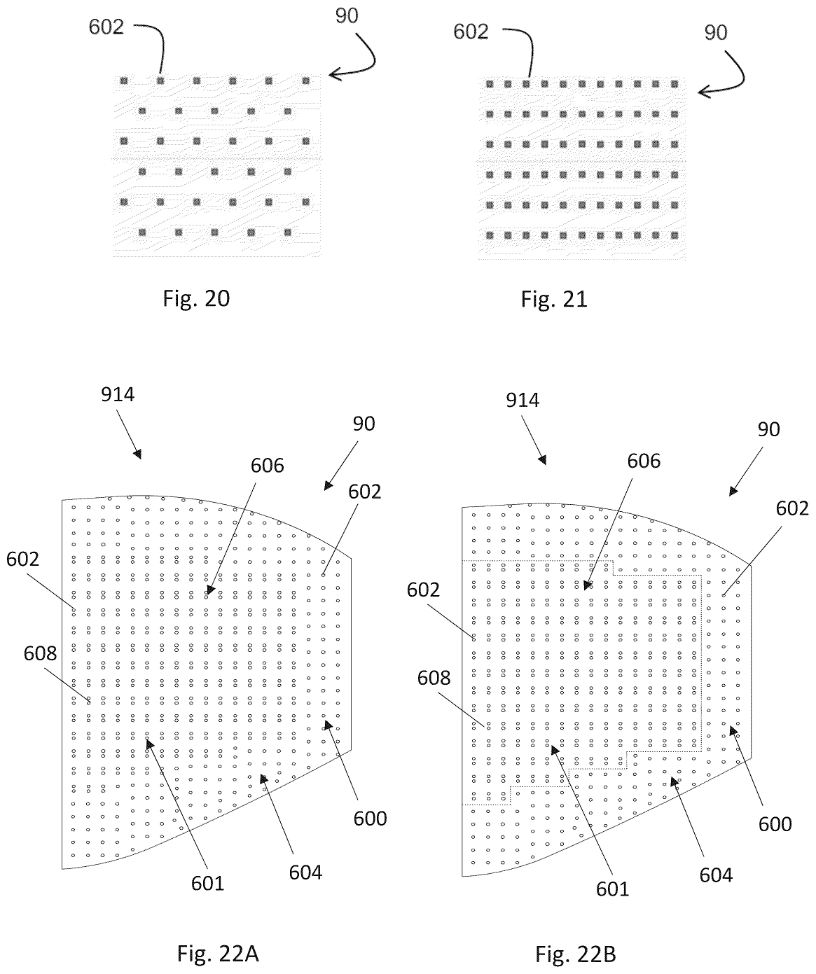

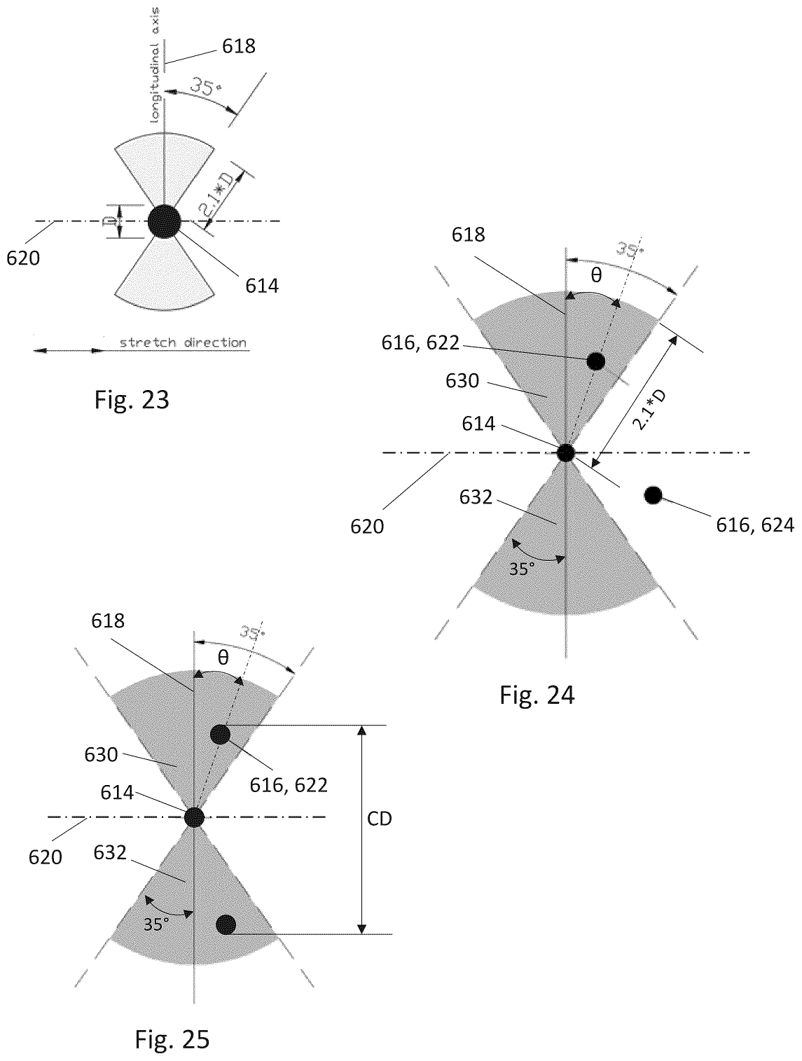

Bond sites are considered to be within a bond site cluster and positioned with respect to one another to form ruptures when each adjacent bond is within about 35 degrees of the longitudinal bond axis and is positioned a distance of less than about 2.1D, where D is the longest bond dimension. More specifically, referring to FIG. 23, a bond site 614 has a longest bond dimension D, which is the longest dimension of the bond site in the direction parallel to the bond longitudinal axis 618. The longest bond dimension, D, may be from about 0.6 mm to about 1 mm or from about 0.6 mm to about 1.5 mm or from about 0.6 mm to about 2 mm or less than about 1 mm or less than about 1.5 mm or less than about 2 mm. For a circular bond, as illustrated in FIG. 23, the longest bond dimension D is the diameter of the primary bond site 614. A bond considered to be within the same bond site cluster and one that would result in stress concentrations that result in aperture formation, should be less than about 2.1D (or 2.1 multiplied by D, the longest bond dimension) and also within about 35 degrees measured from the bond longitudinal axis 618. The dimension of 2.1D is measured from the outer perimeter of the bond site 614, according to the Bond Dimension Test Method.

Referring to FIG. 24, a first or primary bond site 614 may be adjacent to a second bond site 616. Bond sites are adjacent where a straight line can be drawn between the bond sites and the straight line does not intersect another bond. A second bond site 616, also referred to herein as an adjacent bond site, is defined as being longitudinally oriented to a first bond site 614, also referred to herein as a primary bond site, when the first bond site 614 is adjacent to the second bond site 616 and the second bond site 616 is from 0 degrees up to and including 35 degrees from a bond longitudinal axis 618 of the first bond site. The bond longitudinal axis 618 is parallel to the central longitudinal axis 50 of the absorbent article and passes through the centroid, also referred to as the geometric center, of the first bond site 614. The bond longitudinal axis 618 may be substantially perpendicular to the stretch direction of the stretch laminate and perpendicular to the bond transverse axis 620. The bond transverse axis 620 is parallel to the transverse axis 48 of the absorbent article and passes through the centroid of the first bond site 614. As illustrated in FIG. 24, a longitudinally oriented bond site 622 is any bond site located within the shaded region. A transversely oriented bond site 624 is any bond site that is adjacent to the primary bond site and not within 35 degrees of the bond longitudinal axis 618 of the primary bond 614, such as illustrated in FIG. 24. Each adjacent bond to the primary bond has a Bond Separation Angle θ measured from the bond longitudinal axis 618, according to the Bond Measurement Test Method. Stated another way, longitudinally-oriented bonds are those bonds that are adjacent to the primary bond and have a Bond Separation Angle θ from 0 degrees up to and including 35 degrees measured from the bond longitudinal axis 618 of the primary bond site 614. Transversely oriented bonds are those bonds that are adjacent to the primary bond and have a Bond Separation Angle θ greater than 35 degrees. FIG. 24 illustrates a first adjacent bond that is longitudinally oriented and within 2.1D, and a second adjacent bond that it not longitudinally oriented, outside the shaded region. The first adjacent bond and the primary bond may form ruptures in the laminate when stretched. The second adjacent bond will be unlikely to lead to ruptures in the laminate during normal use.

Generally, those bond sites that are longitudinally-oriented bond sites with respect to a primary bond site should be spaced a distance of less than about 2.1D from the primary bond site to form ruptures. The distance between the second bond site and the primary bond site is dependent on the dimension of the primary, first bond site 614. Each primary bond site has a longest bond dimension D, as previously discussed. The longest bond dimension D is the longest dimension of the bond site measured parallel to the bond longitudinal axis 618. For example, as illustrated in FIG. 23, the primary bond site 614 has a longest bond dimension D, which is the longest dimension of the bond site in the direction parallel to the bond longitudinal axis 618. For a circular bond, as illustrated in FIG. 23, the longest bond dimension D is the diameter of the primary bond site 614.

FIG. 24 illustrates zones established by the Bond Separation Angle and the Bond Separation Distance, which is based on the longest bond dimension of the primary bond. An upper zone 630, such as illustrated with shading in FIG. 24, is the zone established by outer circumferential surface of the bond site up to 2.1D for that area within and up to 35 degrees from the bond longitudinal axis 618. A bond located in the upper zone 630 is close enough to the primary bond that stress concentrations will likely be formed during normal use and result in the formation of ruptures. A lower zone 632, such as illustrated with shading in FIG. 24, is the zone extending established by the distance from the outer perimeter of the bond site up to 2.1D for that area up to and including 35 degrees from the bond longitudinal axis 618. The bonds disposed in the lower zone 632 are close enough to the primary bond to form stress concentrations that promote the formation of ruptures during typical use. For bonds positioned outside of the first zone 630 and the second zone 632, these bonds are transversely oriented and have not been found to form stress concentrations with the primary bond 614. Stated another way, bonds oriented transversely to the primary bond 614 are unlikely to form ruptures during typical use of the stretch laminate.

As previously discussed, the rupture form from the bond site cluster should be such that the stretched elastic laminate is breathable and maintains the necessary integrity to function as intended, such as an car for an absorbent article.

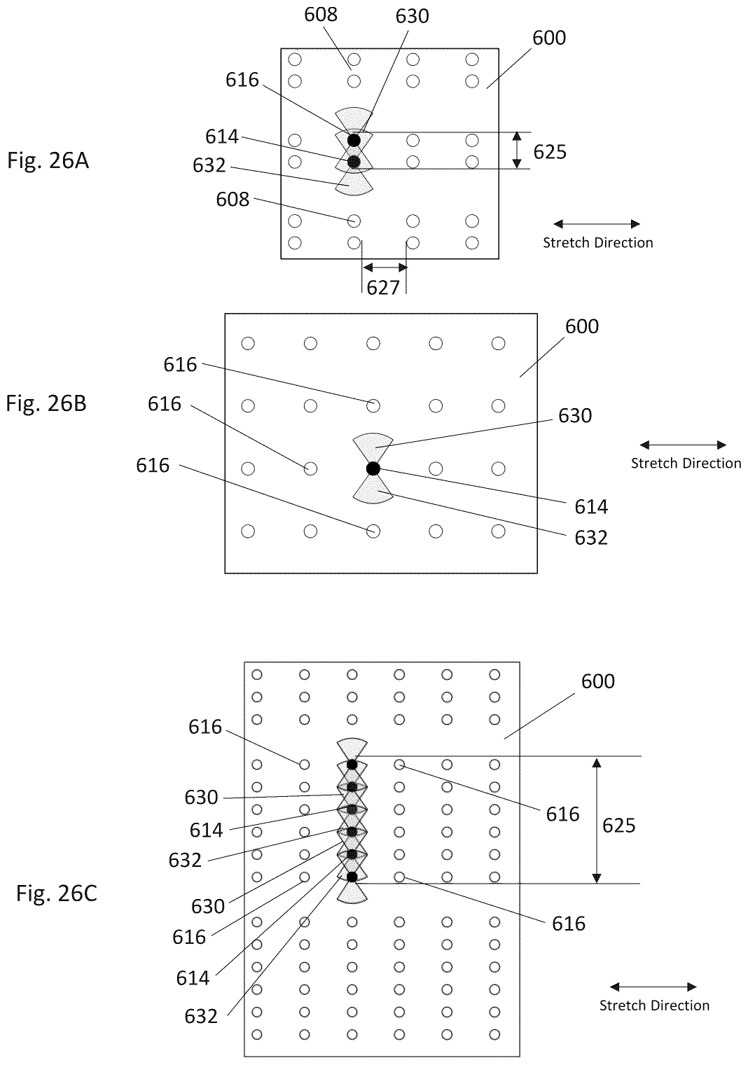

FIGS. 26A-26C illustrate various bond patterns and the various zones based on a primary bond site. For example, FIGS. 26A-26C illustrate bond patterns 600 having a primary bond 614 and a plurality of bonds that are adjacent to the primary bond 614. As illustrated in FIG. 26A, the stretch laminate includes a bond pattern 600 having a plurality of bond site clusters 608. The bond site clusters each include a first bond site 614 and a second bond site 616. As previously discussed, each of the first bond site 614 and the second bond site 616 include an upper zone 630 and a lower zone 632. As illustrated, the second bond site 616 is within the upper zone 630 of the first bond site 614. Thus, the first and second bond site should result in a rupture when the laminate is stretched in the stretch direction during normal use. Each of the clusters of bond sites are separated from each other such that the ruptures will be confined to each of the individual clusters. This prevents large ruptures from resulting in the stretch laminate.

To prevent large ruptures, each of the bond site clusters 608 have a cluster dimension 625 that is less than about 5 mm or less than about 4 mm or less than about 3 mm. The cluster dimension 625 is measured perpendicular to the stretch direction from the outermost point on the upper most bond and the outermost point on the lower most bond within the bond site cluster. FIG. 26A illustrates the cluster dimension 625 for the bond site cluster 608 including a first bond site 614 and a second bond site 616. The cluster dimension 625 may be from about 2 mm to about 5 mm or from about 2 mm to about 4 mm or less than about 5 mm or less than about 3 mm. Restricting the cluster dimension of the bond site cluster results in ruptures that allow for breathability and maintain performance of the stretch laminate. Stated another way, controlling the cluster dimension of the bond site cluster allows for relatively greater breathability when the laminate is in normal use and prevents relatively large tears in the laminate that inhibit the performance of the stretch laminate. It is to be appreciated that any number of bonds may be present within the bond site cluster so long as the individual bonds do not exceed the cluster dimension 625.

Further, each individual bond site cluster needs to be positioned such that the bonds in a first bond site cluster are not within either the upper zone or the lower zone of a second bond site cluster. For example, as illustrated in FIG. 26A, the bond site clusters are positioned at a distance great enough that the rupture does not extend between adjacent bond site clusters but is rather confined to each bond site cluster. Stated another way, each individual cluster may be separated by a cluster separation distance 627, which is the distance from the perimeters of the nearest, adjacent bond sites. The bond site cluster separation distance is at least about 2.1D.

FIG. 26B illustrates a bond pattern that includes bonds that when selected as the primary bond includes adjacent bonds that are outside of both the upper zone 630 and the lower zone 632. Thus, this bond pattern would not result in ruptures due to the spacing of the bonds. FIG. 26C illustrates another bond pattern that includes bonds that are within both the upper zone 630 and the lower zone 632 of adjacent bond sites. However, the number of bond sites exceeds the cluster dimension and, thus, would result in ruptures that would be relatively large and negatively impact the performance of the stretch laminate.

In some embodiments, the bond site clusters may be configured to form ruptures when the stretch laminate is stretched in a stretch direction for greater than about 15 mm or greater than about 20 mm or greater than about 25 mm or greater than about 30 mm, according to the Tensile Test Method disclosed herein.

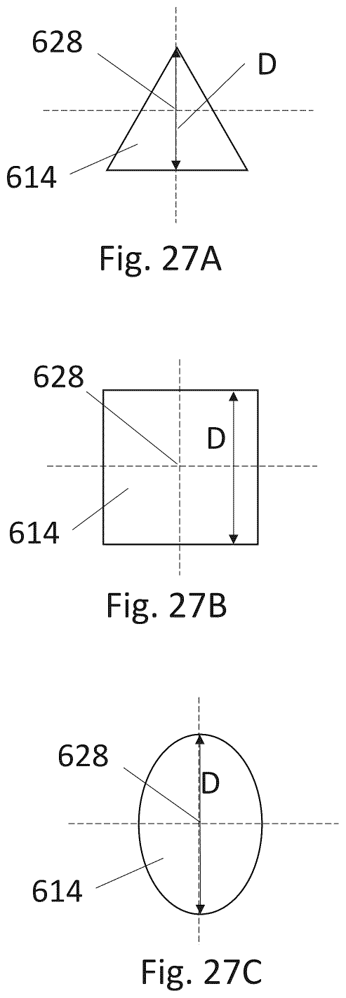

The aforementioned disclosure is also applicable to bonds having non-circular shapes. Independent of the shape of the bond site, the primary bond has a longest bond dimension D measured in a direction parallel to the bond longitudinal axis 618, which is perpendicular to the stretch direction. The longest bond dimension, D, may be from about 0.6 mm to about 1 mm or from about 0.6 mm to about 1.5 mm or from about 0.6 mm to about 2 mm or less than about 1 mm or less than about 1.5 mm or less than about 2 mm. For example, FIGS. 27A-27C illustrate various shapes of bond sites and their longest bond dimension D. As previously described, the longest bond dimension D is the longest dimension of the bond site in the direction parallel to the bond longitudinal axis and is used to determine the upper zone 630 and the lower zone 623.

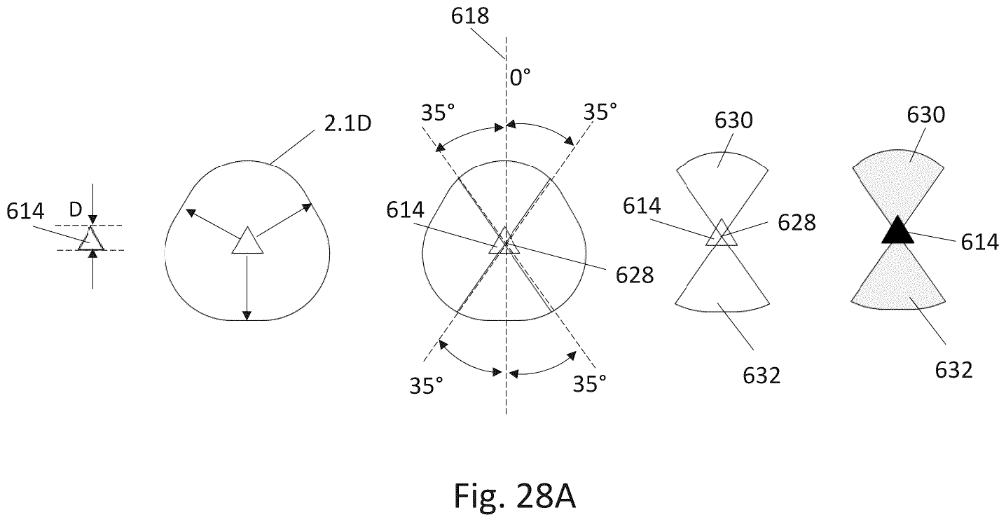

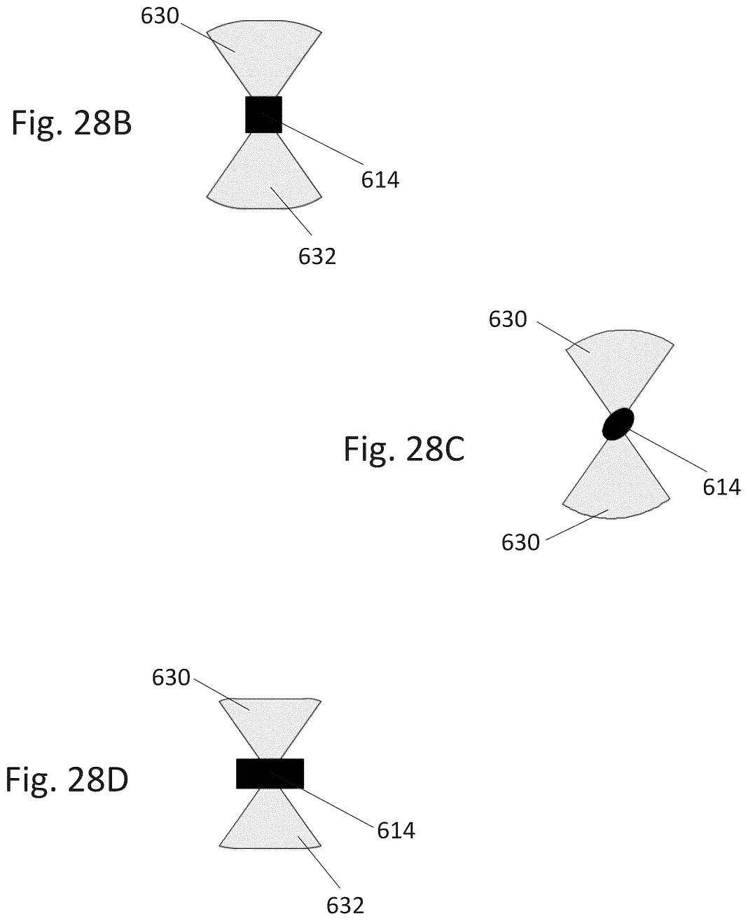

Referring to FIG. 28A, to establish the upper zone 630 and the lower zone 632, the longest bond dimension D of the primary bond site or first bond site 614 is determined. A first boundary may be established by measuring 2.1D from the perimeter of the primary bond site in a direction perpendicular to the perimeter of the primary bond site. The limits of the bond separation angle for longitudinally oriented bonds and laterally oriented bonds are determined. The upper zone 630 including a bond separation angle from 0 degrees up to and including 35 degrees from the bond longitudinal axis 618, can then be determined. The upper zone 630 and the lower zone 632 can then be visually illustrated as shown in FIG. 28A. This method may be used for any bond shape. FIGS. 28B-28D illustrate the upper and lower zones 630, 632 for various other shapes.

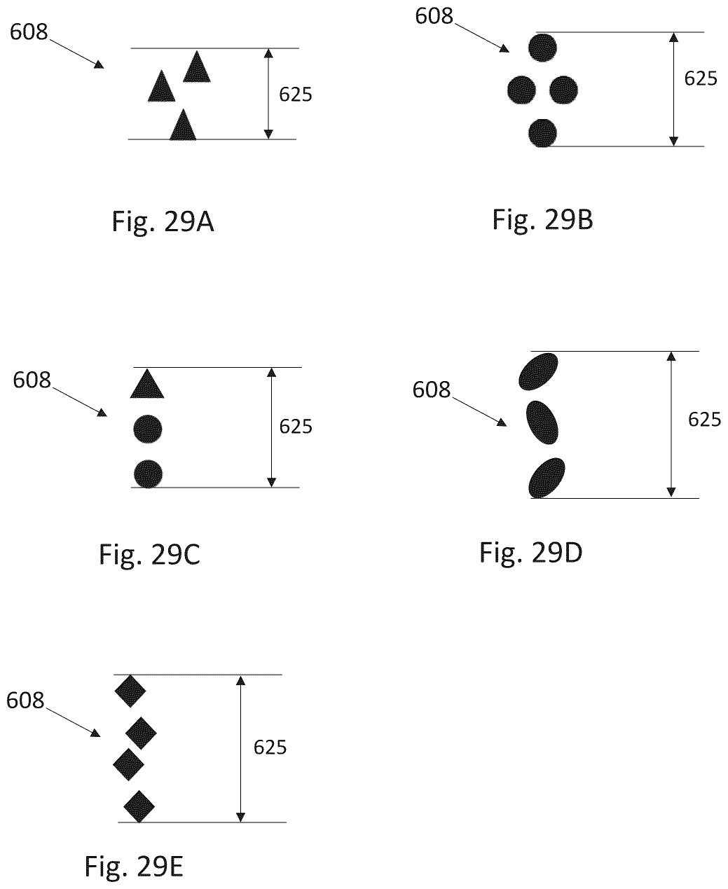

FIGS. 29A-29E illustrate various examples of bond site clusters 608. As previously discussed each of the bond site clusters has a cluster dimension 625. FIG. 29A illustrates a bond site cluster having triangular shaped bonds. FIG. 29B illustrates a bond site cluster having substantially circular-shaped bonds. FIG. 29C illustrates a bond site cluster having two different shaped bonds. FIG. 29D illustrates a bond site cluster having the same shaped bonds with different orientations. FIG. 29E illustrates a bond site cluster having diamond shaped bonds that are unequally spaced in the longitudinal direction, which is perpendicular to the stretch direction. It is to be appreciated that the bond site clusters may include bonds having the same or different shaped bonds and bonds that are equally or unequally spaced with respect to one another.

### Frangible Bonds

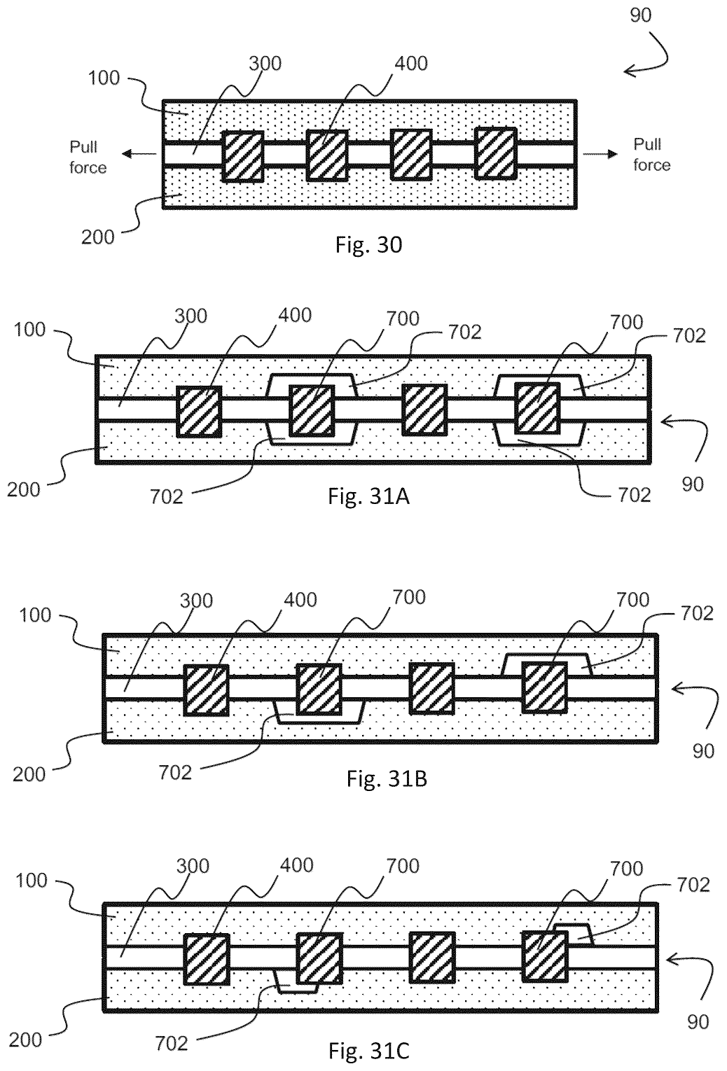

In addition to bond site clusters, the stretch laminate may include one or more frangible bonds. “Frangible bond” refers to a bond that is breakable upon being stretched. FIG. 30 is a schematic illustration of a stretch laminate 90 comprising an elastomeric film layer 300 sandwiched between a first cover layer 100 and a second cover layer 200. The layers are held together with a plurality of ultrasonic bonds 400. The stretch laminate 90 may be subjected to a lateral pull force. FIGS. 31A-31C illustrate a schematic representation of the stretch laminate 90 of FIG. 30 with various types of frangible bond sites 700 after being subjected to the lateral pull force. In FIG. 31A, the frangible bond sites 700 separate from both the first cover layer 100 and the second cover layer 200 at areas of detachment 702. In FIG. 31B, the frangible bond sites 700 separate from either the first cover layer 100 or the second cover layer 200 at areas of detachment 702. In FIG. 31C, the frangible bond sites 700 separate only partially from either the first cover layer 100 or the second cover layer 200 at areas of detachment 702.

### Cover Layer Primary Bond Pattern

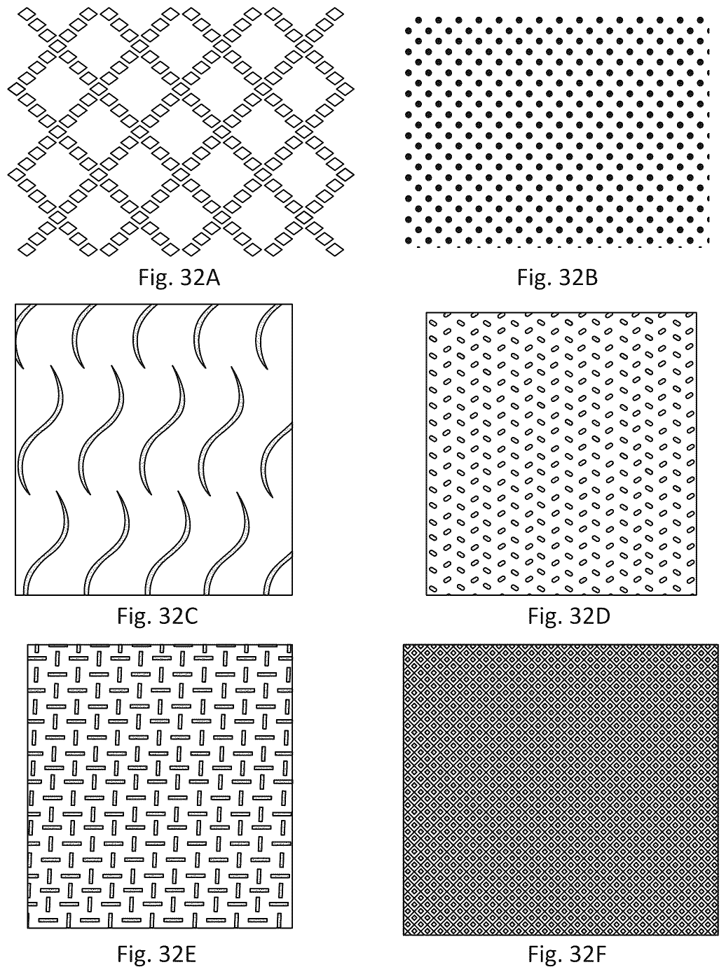

FIG. 32A-32F are schematic illustrations showing a variety of primary bond patterns for the cover layers that may be employed according to various embodiments. The bond patterns may be imprinted or embossed on a cover layer, such as first cover layer 100 or second cover layer 200 as described herein. As described herein, the cover layers may comprise a nonwoven material, in which case the primary bond pattern may be referred to as a nonwoven bond pattern. Such patterns may provide appealing and/or distinctive textures and appearances to stretch laminates.

### Packages

The absorbent articles of the present disclosure may be placed into packages. The packages may comprise cellulose fibers and/or cellulose fiber layers and/or polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

### Test Methods

### Bond Measurement Test Method

The Bond Measurement Test Method is performed on reflected light microscopy images generated using a stereo light microscope (such as Zeiss V20 Stereoscope) and attached camera (such as the Carl Zeiss AxioCam MRc5). The image, containing at least one single repeat unit of a bond impression pattern, is acquired while the sample is fully stretched and backed with a black background. If the area of a single repeat pattern is too large for stereoscope imaging, a DSLR Camera (such as Pentax R20D), or scanner (such as Epson Perfection V750 Pro Flatbed Scanner), capable of at least a 50 micron per pixel resolution may be used to collect the image. Measurements are performed using image analysis software (such as Image Pro Plus software Version 7.0.0.591, Media Cybernetics, USA) calibrated such that distances within the image can be measured precisely to the nearest 50 microns. For purposes of this method, a bond impression is the intentional joining of two or more layers and is the deformed area caused during the bonding process (e.g., the reduced caliper at the site of bonding). Precondition samples at about 23° C.±2° C. and about 50%±2% relative humidity for 2 hours prior to testing under the same environmental conditions.

Prior to and during image acquisition the sample is fully stretched and secured in a planar extended state. For corrugated laminates, the specimen is fully stretched when the corrugations are substantially flattened by extending the laminate while making sure that the inelastic substrates of the laminate are not plastically deformed. For laminates without corrugations, the specimen is considered fully stretched without such extension.

### Bond Separation Distance

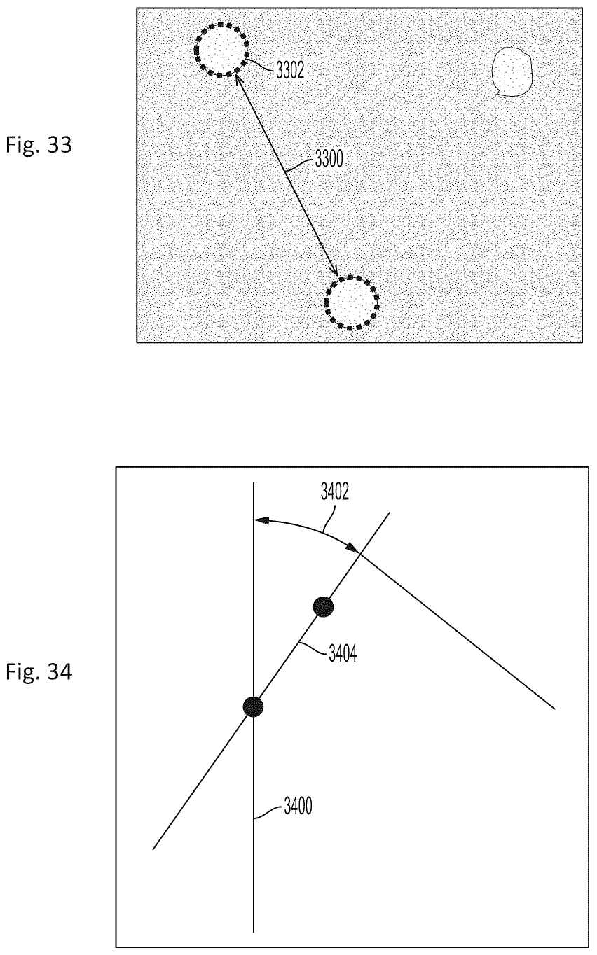

The Bond Separation Distance is defined as the shortest (minimum), straight-line distance 3300 between the perimeters 3302 of any two individual bond sites. Using image analysis software measure and record the Bond Separation Distance for all of the identified the discrete bond sites, as shown for example in FIG. 33. Repeat this procedure for a total of 3 replicates. Calculate and report the arithmetic mean of the recorded values and report as the Bond Separation Distance to the nearest 0.1 mm.

### Bond Separation Angle

For the purposes of this method, the longitudinal axis 3400 is defined as being substantially perpendicular to the primary stretch direction of the laminate. The Bond Separation Angle 3402 between any two bond sites is the angle formed between the bond longitudinal axis 3400 and a line 3404 drawn through the centroids of the two bond sites. Regardless of whether this angle is oriented to the right or left side of the longitudinal axis, the value should be reported as being positive (i.e., the absolute value of the angle). The angle should be chosen such that it is always≤90° from the longitudinal axis. See FIG. 34 for a visual representation. Report this angle to the nearest 1 degree.

### Hysteresis Test Method

The Hysteresis Test can be used to various specified strain or load values. The Hysteresis Test utilizes a commercial tensile tester (e.g., from Instron Engineering Corp. (Canton, MA), SINTECH-MTS Systems Corporation (Eden Prairie, MN) or equivalent) interfaced with a computer. The computer is used to control the test speed and other test parameters and for collecting, calculating, and reporting the data. The tests are performed under laboratory conditions of 23° C.±2° C. and relative humidity of 50%+2%. The specimens are conditioned for 24 hours prior to testing.

Identify a corrugated portion or stretch portion on absorbent article product containing bonds. The specimen is cut from this area of absorbent article product to dimensions listed in the table below for the test performed.

### Test Protocol

1. Select the appropriate grips and load cell. The grips must have one flat surface and must be wide enough to grasp the specimen along its full width. Also, the grips should provide adequate force and suitable surface area to ensure that the specimen does not slip during testing. The grips are air actuated grips designed to concentrate the entire gripping force along a single line perpendicular to the direction of testing stress having one flat surface and an opposing face from which protrudes a half round (radius=6 mm, e.g., part number: 56-163-827 from MTS Systems Corp.) or equivalent grips, to minimize slippage of the specimen. The load cell is selected so that the tensile response from the specimen tested is between 5% and 95% of the capacity of the load cell used. Calibrate the tester according to the manufacturer's instructions.
2. Set the distance between the grips (gauge length) as per the test performed (table below).
3. Place the specimen in the flat surfaces of the grips such that the uniform width lies along a direction perpendicular to the gauge length direction. Sample is mounted in a way that sample stretch direction is the test direction. Secure the specimen in the upper grip, let the specimen hang slack, then close the lower grip.
4. Pre-load: Set the slack pre-load at 0.05N per inch, and pre-load crosshead speed of 13 mm/min. This means that the data collection starts when the slack is removed (at a constant crosshead speed of 13 mm/min) with a force of 0.05N per inch. Strain is calculated based on the adjusted gauge length (lini), which is the length of the specimen in between the grips of the tensile tester at a force of 0.05N per inch. This adjusted gauge length is taken as the initial specimen length, and it corresponds to a strain of 0%. Percent strain at any point in the test is defined as the change in length relative to the adjusted gauge length, divided by the adjusted gauge length, multiplied by 100.
5. a)
   a. First cycle loading: Pull the specimen to the given End Point (load or strain) at a constant cross head speed as defined in the table below for the test. Report the stretched specimen length between the grips as Imax.
   b. First cycle unloading: Hold the specimen at the End Point of step 5 (a) for 30 seconds and then return the crosshead to its starting position (0% strain or initial sample length, lini) at a constant cross head speed defined in step 5 (a) above.
   c. Hold the specimen in the unstrained state for 1 minute.
   d. Second cycle: Repeat Step 5 (a) and 5 (b).

| | Extension and | Hysteresis |
|---|---|---|
| | Tensile Tests | Test |
| Sample Length in the laminate | larger than | >27 |
| stretch direction (mm) | gage length | |
| Sample Width, perpendicular to | 25.4 preferred | 25.4 |
| laminate stretch direction (mm) | (10 mm minimum) | |
| Gauge Length (mm) | Minimum 7 mm, | 25.4 |
| | maximum 25.4 mm | |
| Test Speed (in/min) | 10 | 10 |
| End Point for Step 5(a) | 50% | 4 N/in |

A computer data system records the force exerted on the sample during the test as a function of applied strain. From the resulting data generated, the following quantities are collected and reported.
i. Length of specimen between the grips at a slack preload of 0.05N (lini) to the nearest 0.001 mm.
ii. Length of specimen between the grips on first cycle at the at a given strain or given force (Imax) to the nearest 0.001 mm.
iii. Strain at Imax length is defined as Smax and is calculated as described in the method above.
iv. Length of specimen between the grips at a second cycle load force of 0.05N (lext) to the nearest 0.001 mm.
v. Force at 50% strain during the first load cycle to the nearest 0.01 N/in (reported as Load Force at 50%).
vi. Force at 50% strain during the second unload cycle to the nearest 0.01 N/in (reported as Unload Force at 50%).

% Set is defined as (lext−lini)/(lmax−lini)*100% to the nearest 0.01%.

The testing is repeated for three separate samples and the arithmetic average is reported.

### Tensile Test Method

The Tensile Test is described and illustrated in US Pat. Pub. No. 2018/0042786, titled Array of Absorbent Articles with Ear Portions, by Mueller et al., which is incorporated by reference in its entirety. The Tensile Test is used to measure the strength of a specimen at a relatively high strain rate that represents product application. The method uses a suitable tensile tester such as an MTS 810, available from MTS Systems Corp., Eden Prairie Minn., or equivalent, equipped with a servo-hydraulic actuator capable of speeds exceeding 5 m/s after 28 mm of travel, and approaching 6 m/s after 40 mm of travel. The tensile tester is fitted with a 50 lb. force transducer (e.g., available from Kistler North America, Amherst, N.Y. as product code 9712 B50 (50 lb)), and a signal conditioner with a dual mode amplifier (e.g., available from Kistler North America as product code 5010). Grips should be used to secure the specimens during tensile testing. The opposing grips may have the same width or different widths as specified. All testing is performed in a conditioned room maintained at about 23° C.±2° C. and about 50%±2% relative humidity. Herein, width and length of the specimen are a lateral width and longitudinal length as defined herein. Precondition specimens at about 23° C.±2° C. and about 50%±2% relative humidity for 2 hours prior to testing.

### (a) Grips

The line grips are selected to provide a well-defined gauge and avoid undue slippage. The specimen is positioned such that it has minimal slack between the grips. A portion of one or both grips may be configured to include a material that reduces the tendency of a specimen to slip, (e.g., a piece of urethane or neoprene rubber having a Shore A hardness of between 50 and 70). Six inches wide top and bottom grips are used to clamp the specimen unless specified otherwise.

(b) Tensile Test of Specimen from Absorbent Article-Back Ears

Ears are generally bonded to the chassis via thermal or adhesive or similar bonding. Ears should be separated from the chassis in a way that cars are not damaged, and performance of the car is not altered. If the chassis bond is too strong (i.e., cars will be damaged upon removal), then the portion of the chassis joined to the car should be cut within the chassis material but without damaging the car. Folded fastening systems (e.g., release tapes covering fastening elements) should be unfolded.

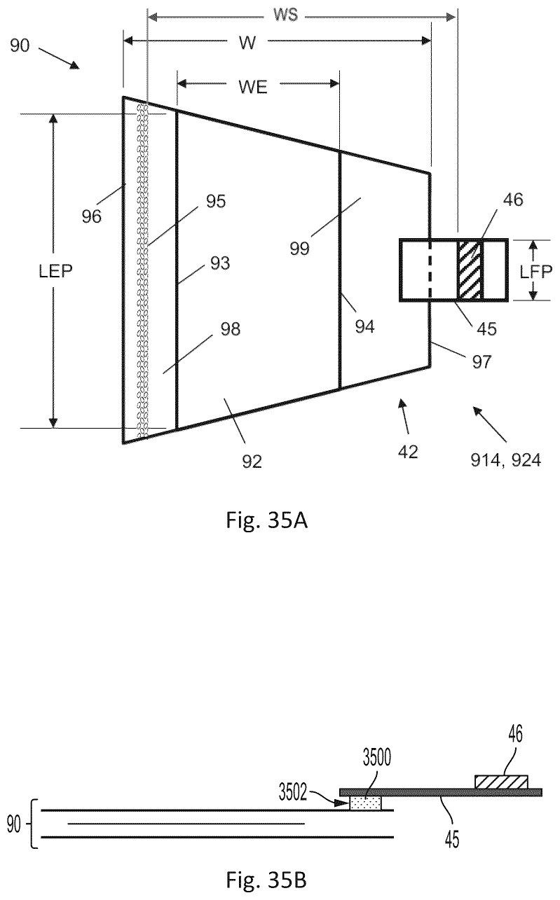

The specimen is clamped in the top grip at a first grip location, which is inboard of the fastener attachment bond 3500, as illustrated in FIGS. 35A and 35B-showing the inboard edge 3502 of the fastener attachment bond 3500. The grip line is kept parallel to the longitudinal centerline of the product. If the fastener attachment bond 3500 is angled, the specimen is gripped at the center of the bond region and grip line is kept parallel to the longitudinal centerline of the product at the center. The width of the top grip should be equal to the maximum length of the fastener attachment bond measured parallel to the longitudinal centerline of the article. If, at the first grip location position, the length of the specimen is the same as the maximum length of the fastener attachment bond, then any grip width greater than the specimen length at the first grip location can be used. The specimen is mounted and hung from the top grip. The opposing edge of the specimen is mounted in the bottom grip in relaxed condition. The bottom grip location is adjusted so the specimen is gripped at the outboard edge of the chassis bond, such as junction line 95 illustrated in FIG. 35A. If the chassis bond is curvilinear, the specimen is gripped at the outboard edge of the outermost bond. The bottom grip is greater than the length of the car at the second grip location. The top and bottom grips are parallel to each other.

The specimen is tested as follows: The vertical distance (perpendicular to the grip line) from the first grip location to the second grip location is measured to 0.1 mm using ruler and is used as gage length for the test. The specimen is tested at a test speed that provides 9.1 sec⁻¹ strain rate with the gage length selected for the specimen. Test speed in mm/second is calculated by multiplying 9.1 sec⁻¹ by the gage length in mm. Before testing, 5 mm of slack is put between the grips.

Each specimen is pulled to break. During testing, one of the grips is kept stationary and the opposing grip is moved. The force and actuator displacement data generated during the test are recorded using a MOOG SmarTEST ONE STO03014-205 standalone controller, with the data acquisition frequency set at 1 KHz. The resulting load data may be expressed as load at break in Newton. The Extension (mm) at 5 N and at 10 N are also recorded. Total of five (5) specimens are run for example. The Average Load at Break and standard deviation, the Average Extension at 5N and standard deviation, and the Average Extension at 10 N and standard deviation are recorded. If, standard deviation recorded is higher than 5%, a new set of five specimens is run.

### (c) Length Ratio-Back Ears

Per the earlier steps, the grips are positioned at a first grip location and a second grip location. The ratio of the length of the specimen at the second grip position (L2) to the first grip position (L1) is Length Ratio. The respective lengths are measured to 0.1 mm accuracy using the ruler.

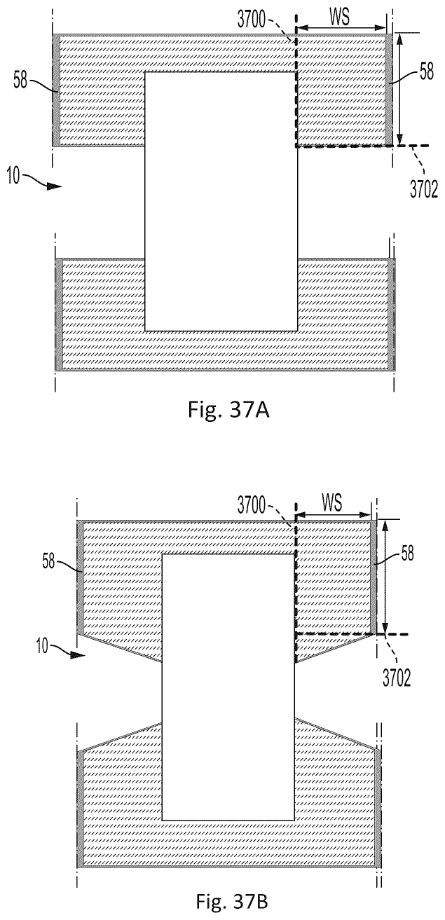

(d) Tensile Test of Specimen from Absorbent Article-Side Members
To prepare belt side member specimens for the Tensile Test:
1. Open the pant 10 by separating the belt side members where they attach to each other, such as the side seam 58 illustrated in FIG. 4. This is performed by cutting through the fused or permanently bonded side seam region 58 with an exacto knife, scissors or equivalent, such as illustrated in FIGS. 37A and 37B. Cut though the side seam such that enough of it remains available for gripping the sample onto the clamps of the tensile tester. In the case of refastenable pants, the side members are not permanently fused or bonded to each other along the lateral sides of the article but instead are attached to each other by releasable fasteners (usually mechanical fastening systems). In these cases, open the pant by carefully disengaging or separating the fasteners that releasably connect the side members to each other along the lateral sides of the article.
2. Cut the belt side member free from the article at a lateral location at the junction line X 3700 so that a grip on the tensile tester can sufficiently grasp the specimen for the testing at the junction line X 3700. The junction line X 3700 is a line parallel to the longitudinal axis of the article that is located at the outboard edge of the absorbent structure. If the absorbent structure is irregularly shaped, the outerboard edge is considered to be the farthest lateral outboard point of the irregularly shaped absorbent structure. Also, cut the belt side member along junction line Y 3702. The junction line Y 3702 is a line parallel to the lateral axis of the article that is located at the longitudinal inboard edge of the side seam such as illustrated in FIGS. 37A and 37B. The junction line Y 3702 may not be a necessary cut if the belt is rectangularly shaped and no longer than the side seam.
2a. In the event, that the pant article 10 contains discrete side members 96 attached to the chassis instead of belts, cut the side member specimen 96 at junction line 95 (consistent with the sampling procedure for Taped diaper back cars). Also, cut the discrete side member along the junction line Y as described herein and continue to step 3.
3. Lay the side member on a substantially flat, horizontal surface and measure width WS with no lateral tension force applied it. WS is distance between the junction line X and the lateral inboard edge of previously separated side seam region 58 as illustrated in FIGS. 37A and 37B.
4. Measure length LFP to the nearest 1 mm, with a steel ruler traceable to NIST or an equivalent. LFP is the longitudinal length of the side member specimen at the outboard cut edge.
5. Mark a midpoint of LFP. The midpoint is half of LFP.
   To test side members for Tensile Testing
1. Insert the outboard edge of the side member into the upper clamp in the tester such that the clamp is centered in the tensile tester fixture and engage the clamp to grip the specimen. Grip the specimen by the side seam region 58 which comprises previously separated permanent side seam attachment bonds or releasable fasteners in the case of refastenable pants. The clamp width is at least as wide as the length of the outboard edge, and preferably is not wider than 1 inch beyond the length of the outboard edge of the specimen. The clamp is set to grip in a location aligned with the outboard edge of the side member within 1 mm, the longitudinal midpoint of the outboard edge of the side member is aligned with the center of the clamp, and the unclamped portion of the side member hangs freely downward from the upper clamp.
2. Insert the inboard edge of the side member into the lower clamp in the tensile tester and in relaxed condition. The lower clamp width is chosen such that no portion of the side member extends beyond the width of the clamp, and preferably the lower clamp width is not wider than 1 inch more than the length of the portion of the side member inserted into the clamp. The clamp is set to grip in a location aligned with the junction line X to within 1 mm, and the specimen is oriented such that if a lateral line perpendicular to a longitudinal axis of the pant having the side members were drawn from the midpoint of LFP, it would extend vertically and align with the center of the fixture holding the lower clamp.

The specimen is tested as follows: The vertical distance (perpendicular to the grip line) from the first grip location to the second grip location is measured to 0.1 mm using ruler and is used as gage length for the test. The specimen is tested at a test speed that provides 9.1 sec⁻¹ strain rate with the gage length selected for the specimen. Test speed in mm/second is calculated by multiplying 9.1 sec⁻¹ by the gage length in mm. Before testing, 5 mm of slack is put between the grips.

Each specimen is pulled to break. During testing, one of the grips is kept stationary and the opposing grip is moved. The force and actuator displacement data generated during the test are recorded using a MOOG SmarTEST ONE STO03014-205 standalone controller, with the data acquisition frequency set at 1 KHz. The resulting load data may be expressed as load at break in Newton. The Extension (mm) at 5 N and at 10 N are also recorded. Total of five (5) specimens are run for example. The Average Load at Break and standard deviation, the Average Extension at 5N and standard deviation, and the Average Extension at 10 N and standard deviation are recorded. If, standard deviation recorded is higher than 5%, a new set of five specimens is run.

### Back Ear Hang Time Test

The back ear hang test is used to determine the resistance of a back car 42 to rupture when exposed to an applied force over a period. Conduct the test in a temperature-controlled room at 38° C.±2° C. and 18%±2% relative humidity which is referred to herein as the test environment. Condition the samples in test environment for at least 2 hours prior to testing.

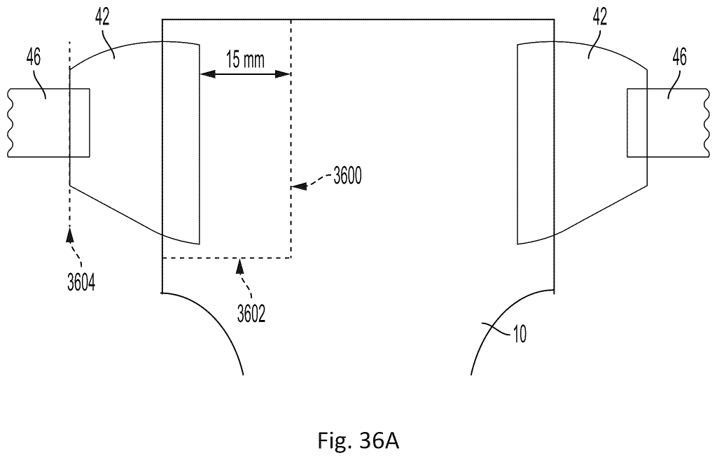

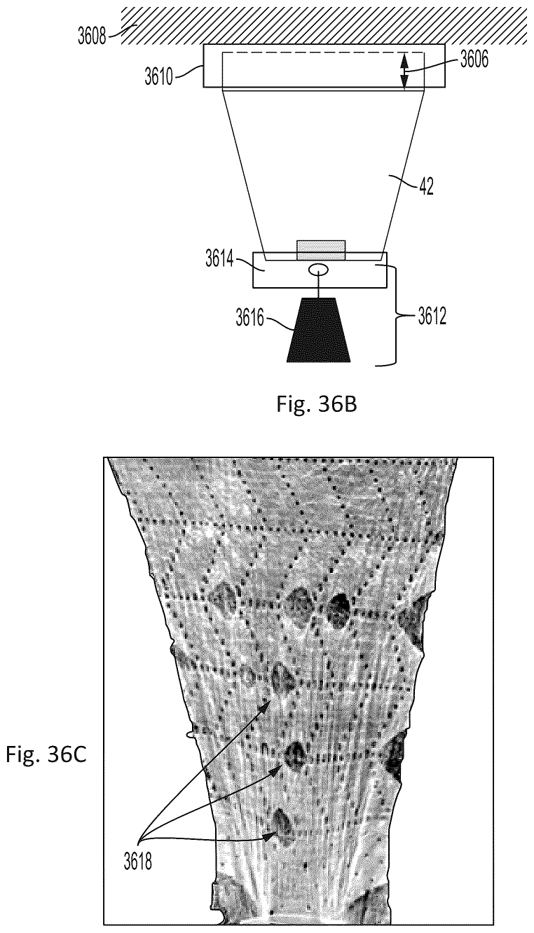

To prepare a test sample, unfold a taped diaper 10, identify the back cars 42. Do not extend the laminate or disrupt the natural relaxed state of the back cars 42. Remove both back cars 42 from the diaper 10 by creating a straight-line incision (cut line A 3600) 15 mm from the inboard edge of the back car laminate 42 and another straight-line incision (cut line B 3602) just below the back car laminate 42. This 15 mm of material extending beyond the inboard edge 3606 of the back car 42 will be subsequently used for attachment to the rigid surface 3608. Use scissors, exacto knife or equivalent to remove the back cars 42. For both back cars 42, also remove the fastening tape 45 portion that is not attached to the chassis by creating a straight-line incision (cut line C 3604) just outside of the outboard edge of the back car laminate. (See, for example, FIG. 36A).

Secure the 15 mm of material beyond the inboard edge of the back car test sample 3606 to a rigid surface 3608 in such a way that the fastening tape 45 edge hangs vertically and freely. Use clamps 3610, tape, or equivalent fixture that is at least as wide as the outboard edge of the back car 42 to secure the back car 42 to the rigid surface 3608 without slippage.

Prepare a weight-assembly 3612 that can be adapted to hang vertically from the fastening tape edge. This weight-assembly 3612 consists of an attachment fixture 3614 and a hanging weight 3616 whose combined mass is 1035±2 grams. The attachment fixture 3614 connects the weight 3616 to the fastening tape 45. The attachment fixture 3614 must be at least as wide as the width of the fastening tape 45 (see, for example, FIG. 36B). The weight-assembly 3612 must be centered with the fastening tape edge, it must be attached securely enough to prevent slippage during the test and the hanging weight 3616 must be allowed to do so freely without contacting any other object or surface.

Attach the weight-assembly 3612 to the fastening tape edge and start a timer at the same time the weight assembly 3612 is released to hang freely. If a flaw in the test sample is present causing an immediate appearance of a rupture, discard both test samples and prepare another set. Allow the sample to remain undisturbed under the applied force of the weight assembly for 3 hours in the test environment. At the end of the 3 hours, the sample will be inspected with the weight-assembly still attached. Visually inspect the back car sample and record the absence or presence of a rupture 3618 (apertures, slits, holes, or tears) of any shape in the film (see, for example, FIG. 36C). A rupture is defined as occurring in at least one of the cover layer and the film layer whose longest dimension in any direction is greater than 1 mm. Repeat this procedure for both back car test samples, if a rupture is identified in either of the back car test samples then record the result as the presence of ruptures.

### Further Definitions and Cross-References

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

### Combinations

A. A stretch laminate for an absorbent article comprising: a nonwoven substrate; and an elastomeric film joined to the nonwoven substrate by a plurality of ultrasonic bonds, wherein the plurality of ultrasonic bonds form a bond pattern comprising: a plurality of bond site clusters, wherein each of the bond site clusters comprise: a first bond having a longest bond dimension, D; and a second bond adjacent to the first bond; wherein a Bond Separation Distance between the first and second bond is less than 2.1D and a Bond Separation Angle between the first bond and the second bond is from 0° up to and including 35° from the bond longitudinal axis, wherein the first bond has a first outermost point and the second bond has a second outermost point, wherein a longitudinal cluster length measured in a direction substantially perpendicular to the stretch direction from the first outermost point to the second outermost point is less than about 5 mm.
B. The bond pattern according to paragraph A, wherein the longitudinal cluster length is less than about 4 mm.
C. The stretch laminate according to any one of the preceding paragraphs, wherein each of the plurality of bond site clusters comprises: a third bond positioned between the first bond and the second bond, wherein the Bond Separation Distance between the first bond and the third bond is less than 2.1D and a Bond Separation Angle between the first bond and the third bond is from 0° up to and including 35° from the bond longitudinal axis, and wherein the Bond Separation Distance between the second bond and the third bond is less than 2.1D and a Bond Separation Angle between the second bond and the third bond is from 0° up to and including 35° from the bond longitudinal axis.
D. The stretch laminate according to paragraph C, wherein the plurality of bond site clusters comprises: a fourth bond positioned between the first bond and the second bond, wherein the Bond Separation Distance between the fourth bond and each adjacent bond is less than 2.1D and a Bond Separation Angle between the fourth bond and each adjacent bond is from 0° up to and including 35° from the bond longitudinal axis.
E. The stretch laminate according to any one of the preceding paragraphs, where the value of D is in the range of about 0.6 mm to about 1.0 mm.
F. The stretch laminate according to any one of the preceding paragraphs, wherein the bonds are at least one of circular, ovaloid, and rectangular in shape.
G. The stretch laminate according to any one of the preceding paragraphs, wherein the laminate has a Load Force at 50% of about 0.3 N/in of greater, according to the Hysteresis Test Method.
H. The stretch laminate according to paragraph G, wherein the laminate has a Load Force at 50% of about 0.45 N/in to about 2 N/in, according to the Hysteresis Test Method.
I. The stretch laminate according to any one of the preceding paragraphs, wherein the laminate has a Smax of about 50% or greater, according to the Hysteresis Test Method.
J. The stretch laminate according to paragraph I, wherein the laminate has a Smax from about 100% to 500%, according to the Hysteresis Test Method.
K. The stretch laminate according to any one of the preceding paragraphs, wherein the stretch laminate includes both an elastic region and an inelastic region, wherein the plurality of ultrasonic bonds are disposed in the elastic region.
L. An absorbent article comprising the stretch laminate of any one of the preceding paragraphs.
M. The absorbent article according to paragraph L, wherein the stretch laminate forms a side panel or car.
N. The absorbent article according to paragraph L, wherein the stretch laminate forms a central waist element.
AA. An absorbent article comprising: a stretch laminate, the stretch laminate comprising: a nonwoven material; an elastomeric film joined to the nonwoven material by a plurality of ultrasonic bonds, wherein the plurality of ultrasonic bonds form a bond pattern, wherein the plurality of ultrasonic bonds comprise a plurality of individual bonds each having a longest bond dimension, D, in the range of about 0.6 mm to about 1.5 mm, wherein the plurality of ultrasonic bonds comprising: a plurality of bond site clusters, wherein each of the bond site clusters comprise: a first bond; and a second bond adjacent to the first bond; wherein a Bond Separation Distance between the first and second bond is less than 2.1D and a Bond Separation Angle between the first bond and the second bond is from 0° up to and including 35° from the bond longitudinal axis, and wherein the each of the plurality of bond site clusters is separated by a cluster separation distance, wherein the cluster separation distance is at least about 2.1D.
BB. The absorbent article according to paragraph AA, wherein each of the plurality of bond site clusters comprises: a third bond positioned between the first bond and the second bond, wherein the Bond Separation Distance between the first bond and the third bond is less than 2.1D and the Bond Separation Angle between the first bond and the third bond is from 0° up to and including 35° from the bond longitudinal axis, and wherein the Bond Separation Distance between the second bond and the third bond is less than 2.1D and the Bond Separation Angle between the second bond and the third bond is from 0° up to and including 35° from the bond longitudinal axis.
CC. The absorbent article according to any one of the preceding paragraphs, wherein the stretch laminate comprises a second nonwoven material, wherein the elastomeric film is positioned intermediate the nonwoven material and the second nonwoven material, wherein the at least some of the plurality of ultrasonic bonds do not overlap the elastomeric film and only join the nonwoven material and the second nonwoven material.
DD. The absorbent article according to any one of the preceding paragraphs, wherein the stretch laminate forms a side panel or car disposed in a waist region of the absorbent article.
EE. The absorbent article according to any one of the preceding paragraphs, wherein the stretch laminate forms a central waist element.
FF. The absorbent article according to paragraph EE, wherein the central waist element is an elastic waist feature in a front waist region or back waist region of the absorbent article.
GG. The absorbent article according to paragraph FF, comprising an acquisition layer between a topsheet and an absorbent core, and wherein a portion of the acquisition layer overlaps a portion of the elastic waist feature.
HH. The absorbent article according to paragraph FF, comprising a core bag between a topsheet and a backsheet, and wherein a portion of the core bag overlaps a portion of the elastic waist feature.
II. The absorbent article according to paragraph GG, wherein the absorbent core is shaped.
JJ. The absorbent article according to paragraph GG, wherein the absorbent article has a front waist region, a back waist region, and a central region between the front waist region and the back waist region, and wherein the absorbent core is wider in at least one of the front waist region and the back waist region than in the central region.
KK. A stretch laminate for an absorbent article, the stretch laminate comprising: a nonwoven material; an elastomeric film joined to the nonwoven material by a plurality of ultrasonic bonds, wherein the plurality of ultrasonic bonds form a bond pattern, wherein the plurality of ultrasonic bonds comprise a plurality of individual bonds each having a longest bond dimension, D, in the range of less than about 1.0 mm, wherein the plurality of ultrasonic comprising: a plurality of bond site clusters, wherein each of the bond site clusters comprise: a first bond having a longest bond dimension, D; and a second bond adjacent to the first bond; wherein a Bond Separation Distance between the first and second bond is less than 2.1D and a Bond Separation Angle between the first bond and the second bond is from 0° up to and including 35° from the bond longitudinal axis, and wherein each of the bond site clusters are configured to form a break in the elastomeric film when the stretch laminate is stretched greater than about 15 mm in a stretch direction.

What is claimed is:

1. A stretch laminate for an absorbent article comprising:
a nonwoven substrate; and
an elastomeric film joined to the nonwoven substrate by a plurality of ultrasonic bonds, wherein the plurality of ultrasonic bonds form a bond pattern comprising:
a plurality of bond site clusters, wherein each of the bond site clusters comprise:
a first bond having a longest bond dimension, D; and
a second bond adjacent to the first bond;
wherein a Bond Separation Distance between the first and second bond is less than 2.1D and a Bond Separation Angle between the first bond and the second bond is from 0° up to and including 35° from the bond longitudinal axis,
wherein the first bond has a first outermost point and the second bond has a second outermost point, wherein a longitudinal cluster length measured in a direction substantially perpendicular to the stretch direction from the first outermost point to the second outermost point is less than 5 mm.

2. The bond pattern of claim 1, wherein the longitudinal cluster length is less than 4 mm.

3. The stretch laminate of claim 1, wherein each of the plurality of bond site clusters comprises:
a third bond positioned between the first bond and the second bond, wherein the Bond Separation Distance between the first bond and the third bond is less than 2.1D and a Bond Separation Angle between the first bond and the third bond is from 0° up to and including 35° from the bond longitudinal axis, and
wherein the Bond Separation Distance between the second bond and the third bond is less than 2.1D and a Bond Separation Angle between the second bond and the third bond is from 0° up to and including 35° from the bond longitudinal axis.

4. The stretch laminate of claim 3, wherein the plurality of bond site clusters comprises:
a fourth bond positioned between the first bond and the second bond, wherein the Bond Separation Distance between the fourth bond and each adjacent bond is less than 2.1D and a Bond Separation Angle between the fourth bond and each adjacent bond is from 0° up to and including 35° from the bond longitudinal axis.

5. The stretch laminate of claim 1, where the value of D is in the range of about 0.6 mm to about 1.0 mm.

6. The stretch laminate of claim 1, wherein the bonds are at least one of circular, ovaloid, and rectangular in shape.

7. The stretch laminate of claim 1, wherein the laminate has a Load Force at 50% of 0.3 N/in of greater, according to the Hysteresis Test Method.

8. The stretch laminate of claim 1, wherein the laminate has a Sₘₐₓ of 50% or greater, according to the Hysteresis Test Method.

9. The stretch laminate of claim 1, wherein the stretch laminate includes both an elastic region and an inelastic region, wherein the plurality of ultrasonic bonds are disposed in the elastic region.

10. An absorbent article comprising:
a stretch laminate, the stretch laminate comprising:
a nonwoven material;
an elastomeric film joined to the nonwoven material by a plurality of ultrasonic bonds, wherein the plurality of ultrasonic bonds form a bond pattern, wherein the plurality of ultrasonic bonds comprise a plurality of individual bonds each having a longest bond dimension, D, in the range of about 0.6 mm to about 1.5 mm,
wherein the plurality of ultrasonic bonds comprising:
a plurality of bond site clusters, wherein each of the bond site clusters comprise:
a first bond; and
a second bond adjacent to the first bond;
wherein a Bond Separation Distance between the first and second bond is less than 2.1D and a Bond Separation Angle between the first bond and the second bond is from 0° up to and including 35° from the bond longitudinal axis, and
wherein the each of the plurality of bond site clusters is separated by a cluster separation distance, wherein the cluster separation distance is at least 2.1D.

11. The absorbent article of claim 10, wherein each of the plurality of bond site clusters comprises:
a third bond positioned between the first bond and the second bond, wherein the Bond Separation Distance between the first bond and the third bond is less than 2.1D and the Bond Separation Angle between the first bond and the third bond is from 0° up to and including 35° from the bond longitudinal axis, and
wherein the Bond Separation Distance between the second bond and the third bond is less than 2.1D and the Bond Separation Angle between the second bond and the third bond is from 0° up to and including 35° from the bond longitudinal axis.

12. The absorbent article of claim 10, wherein the stretch laminate comprises a second nonwoven material, wherein the elastomeric film is positioned intermediate the nonwoven material and the second nonwoven material, wherein the at least some of the plurality of ultrasonic bonds do not overlap the elastomeric film and only join the nonwoven material and the second nonwoven material.

13. The absorbent article of claim 10, wherein the stretch laminate forms a side panel or ear disposed in a waist region of the absorbent article.

14. The absorbent article of claim 10, wherein the stretch laminate forms a central waist element.

15. The absorbent article of claim 14, wherein the central waist element is an elastic waist feature in a front waist region or back waist region of the absorbent article.

16. The absorbent article of claim 15, comprising an acquisition layer between a topsheet and an absorbent core, and wherein a portion of the acquisition layer overlaps a portion of the elastic waist feature.

17. The absorbent article of claim 15, comprising a core bag between a topsheet and a backsheet, and wherein a portion of the core bag overlaps a portion of the elastic waist feature.

18. The absorbent article of claim 16, wherein the absorbent core is shaped.

19. The absorbent article of claim 16, wherein the absorbent article has a front waist region, a back waist region, and a central region between the front waist region and the back waist region, and wherein the absorbent core is wider in at least one of the front waist region and the back waist region than in the central region.

20. A stretch laminate for an absorbent article, the stretch laminate comprising:
a nonwoven material;
an elastomeric film joined to the nonwoven material by a plurality of ultrasonic bonds, wherein the plurality of ultrasonic bonds form a bond pattern, wherein the plurality of ultrasonic bonds comprise a plurality of individual bonds each having a longest bond dimension, D, in the range of less than about 1.0 mm,
wherein the plurality of ultrasonic comprising:
a plurality of bond site clusters, wherein each of the bond site clusters comprise:
a first bond having a longest bond dimension, D; and
a second bond adjacent to the first bond;
wherein a Bond Separation Distance between the first and second bond is less than 2.1D and a Bond Separation Angle between the first bond and the second bond is from 0° up to and including 35° from the bond longitudinal axis, and
wherein each of the bond site clusters are configured to form a break in the elastomeric film when the stretch laminate is stretched greater than 15 mm in a stretch direction.
